(19) United States
(12) Patent Application Publication
(10) Pub. No.: US 2026/0351026 A1
(43) Pub. Date: Nov. 5, 2026

(54) Method, apparatus, and system for data acquisition

(71) Applicants: BGI SHENZHEN, Shenzhen (CN); BGI SHENZHEN CO., LTD, Shenzhen (CN)

(72) Inventors: Chao Liu, Shenzhen (CN); Tianyi Xia, Shenzhen (CN); Chang Shi, Shenzhen (CN); Junfu Guo, Shenzhen (CN); Yong Peng, Shenzhen (CN); Bohan Zhang, Shenzhen (CN); Qin Lu, Shenzhen (CN); Mei Li, Shenzhen (CN); Yong Zhang, Shenzhen (CN); Yuxiang Li, Shenzhen (CN)

(21) Appl. No.: 19/487,262

(22) PCT Filed: May 24, 2023
(86) PCT No.: PCT/CN2023/096110

§ 371 Date: Nov. 24, 2025

(51) Int. Cl. G06V 20/69; G06T 5/40; G06T 5/50; G06T 7/00; G06T 7/33; G06T 17/00; G06V 10/46; G16H 30/20; G16H 30/40

(52) U.S. Cl. CPC G06V 20/695; G06T 5/40; G06T 5/50; G06T 7/0012; G06T 7/33; G06T 17/00; G06V 10/46; G16H 30/20; G16H 30/40; G06T 2207/20221; G06T 2207/30024

(57) ABSTRACT

Embodiments of the present disclosure provide a method, apparatus, and system for data acquisition, which relate to the technical field of data processing. The method comprises: sectioning a biological tissue sample, and performing time-and-space omics sequencing on the sections to obtain expression level data; performing image acquisition on the sectioned biological tissue sample to obtain image data; performing registration on the expression level data and/or the image data to generate an outer-layer contour and/or a partition contour of the biological tissue sample; and generating three-dimensional display data for the biological tissue sample according to the outer-layer contour and/or the partition contour. The present disclosure provides a whole set of apparatus and method for data acquisition, and a registration method for expression level data and tissue section image data of time-and-space omics sequencing. Aiming at in situ capture time-and-space omics technology, combined with optical imaging information and assistance of rigid and elastic registration of continuous tissue sections, three-dimensional reconstruction is achieved; therefore, auxiliary visual information positioning of inner and outer tissue contours is obtained, and time-and-space omics data with large size and high resolution is effectively processed and displayed.

### CROSS-REFERENCE TO RELATED APPLICATION

This application is a U.S. national stage application of International Application No. PCT/CN2023/096110, filed on May 24, 2023, the entire contents of which are incorporated herein by reference.

### FIELD

The present disclosure relates to the technical field of data processing, in particular to a data acquisition method, apparatus, and system.

### BACKGROUND

Since the development of spatiotemporal omics technology, it has been preliminarily applied in biology fields such as neuroscience, pathology, and developmental biology. Spatiotemporal omics analysis on a two-dimensional plane makes it difficult to obtain three-dimensional morphological structure and accurate positional information. It is of considerable technical challenge and significant scientific importance to figure out how to overcome the limitations of wo-dimensional plane and enable three-dimensional spatiotemporal omics research. At present, the research focuses on three-dimensional reconstruction methods for spatiotemporal omics, aiming to achieve comprehensive analysis of the spatial distribution of different cells, cell-cell interactions, expression of genes and proteins, and overall morphological structure at the three-dimensional level. The current three-dimensional reconstruction of spatiotemporal omics performs rigid registration on the expression data that is only the abundance information of expression captured by spatiotemporal omics on the 2D plane. However, problems with this approach include low accuracy in reconstructing data back to the in-situ expression, limited visualization effects, and insufficient capability to process large-scale tissue sample data and conduct post-processing display.

### SUMMARY

The embodiments of the present disclosure propose a data acquisition method, apparatus, and system, which combines the in-situ captured spatiotemporal omics technology with optical imaging information, to obtain internal and external tissue contours for assisting the positioning of visualization information. From this, position registration at the cellular resolution for each information collection site, and effective processing and display for large-scale and high-resolution spatiotemporal omics data are achieved.

According to a first aspect of embodiments of the present disclosure, there is provided a data acquisition method, including: sectioning a biological tissue sample and performing spatiotemporal omics sequencing on a section to obtain expression data; acquiring an image of a sectioned biological tissue sample to obtain image data; registering at least one of the expression data and the image data to generate at least one of an external contour and a region contour of the biological tissue sample; generating a three-dimensional display data of the biological tissue sample based on at least one of the external contour and the region contour.

In some embodiments, wherein said sectioning a biological tissue sample and performing spatiotemporal omics sequencing on a section to obtain expression data includes: performing serial sectioning on the biological tissue sample, and performing spatiotemporal omics sequencing upon sectioning a first predetermined number of sections, to obtain the expression data of the sections, wherein the first predetermined number is a positive integer.

In some embodiments, the method further includes performing preprocessing on the expression data, wherein the preprocessing includes at least one of aggregation processing, quality control, logarithmic normalization, highly variable gene identification, or dimensionality reduction.

In some embodiments, wherein said acquiring an image of the sectioned biological tissue sample to obtain image data includes: performing a single image acquisition on the sectioned biological tissue sample upon sectioning a second predetermined number of sections, to obtain the image data, wherein the second predetermined number is a positive integer.

In some embodiments, wherein said performing registration on at least one of the expression data and the image data to generate at least one of an external contour and region contour of the biological tissue sample includes: performing an image segmentation on the image data to generate a segmented image; registering the segmented image to generate a registered image; generating the external contour of the biological tissue sample based on the registered image.

In some embodiments, wherein said performing an image segmentation on the image data includes: converting the image data to HSV color space and extracting image data of H-channel; converting the image data of H-channel to a grayscale image; binarizing the image data using a preset image processing algorithm to determine a binary contour image; retaining the binary contour image meeting a preset condition and merging the retained binary contour image with the image data.

In some embodiments, the method further includes: obtaining a grayscale image and obtaining a histogram of the image data based on the grayscale image; binarizing the image data using a maximum entropy threshold; merging the binary image with the image data.

In some embodiments, wherein said registering the segmented image to generate a registered image includes: performing a processing of a spatial transformation and a linear interpolation algorithm on the segmented image; measuring a mean squared error similarity between the segmented image after processing and a reference image of registration; determining an optimal measurement solution as the registered image.

In some embodiments, wherein said generating the external contour of the biological tissue sample based on the registered image includes: stacking all registered images into an array file with a preset format; processing the array file to obtain the external contour of the biological tissue sample.

In some embodiments, wherein said performing registration on at least one of the expression data and the image data to generate an external contour and/or region contour of the biological tissue sample includes: calculating the expression data to obtain an expression image; performing a histogram equalization on the expression image and the image data to obtain an image pair of the expression image-the image data; performing a rigid registration and an elastic registration in sequence for the image pair to generate the region contour.

In some embodiments, wherein said performing a rigid registration for the image pair includes: downsampling the image pair respectively; extracting feature information from the image pair; performing a similarity-estimated affine transformation on the image pair to obtain a rigid registration result.

In some embodiments, wherein said performing an elastic registration on the image pair includes: performing a histogram equalization on the rigid registration result; inputting the rigid registration result into a deep learning model to output an intermediate result by the deep learning model, wherein the intermediate result is a deformation field, wherein the deep learning model is trained using the image pair of the expression image-the image data; performing nearest-neighbor interpolation transformation on the rigid registration result based on the deformation field to obtain an elastic registration result.

In some embodiments, wherein said performing registration on at least one of the expression data and the image data to generate at least one of an external contour and region contour of the biological tissue sample includes: registering expression data from two adjacent or proximate sections to obtain registered expression data; generating the external contour and/or the region contour of the biological tissue sample based on the registered expression data.

In some embodiments, wherein said generating a three-dimensional display data of the biological tissue sample based on at least one of the external contour and the region contour includes: for the registration result obtained by performing registration on at least one of the expression data and the image data, performing bioinformatics analysis to obtain clustering and annotation results; constructing a three-dimensional array based on the clustering and annotation results, and incorporating spatial scale information; rendering with a three-dimensional rendering tool, to obtain the three-dimensional display data of the biological tissue sample.

According to a second aspect of embodiments of the present disclosure, there is provided a data processing method, including: acquiring expression data and image data of a biological tissue sample, wherein the expression data is obtained by sectioning the biological tissue sample and performing spatiotemporal omics sequencing on a section, and the image data is obtained by acquiring an image of a sectioned biological tissue sample; calculating the expression data to obtain an expression image; performing a histogram equalization on the expression image and the image data to obtain an image pair of the expression image-the image data; performing a rigid registration and an elastic registration in sequence for the image pair to generate contour data of the biological tissue sample, wherein the contour data is used to generate the three-dimensional display data of the biological tissue sample.

In some embodiments, wherein said performing a rigid registration on the image pair includes: downsampling the image pair respectively; extracting feature information from the image pair; performing a similarity-estimated affine transformation on the image pair to obtain a rigid registration result.

In some embodiments, wherein said performing an elastic registration on the image pair includes: performing a histogram equalization on the rigid registration result; inputting the rigid registration result into a deep learning model to output an intermediate result by the deep learning model, wherein the intermediate result is a deformation field, wherein the deep learning model is trained using the image pair of the expression image-the image data; performing nearest-neighbor interpolation transformation on the rigid registration result based on the deformation field to obtain an elastic registration result.

According to a third aspect of embodiments of the present disclosure, there is provided a data acquisition apparatus, including: a sequencing module, configured to section a biological tissue sample and perform spatiotemporal omics sequencing on a section to obtain expression data; an acquisition module, configured to acquire an image of a sectioned biological tissue sample to obtain image data; a registration module, configured to perform a registration on the expression data and/or the image data to generate an external contour and/or region contour of the biological tissue sample; a generation module, configured to generate three-dimensional display data of the biological tissue sample based on the external contour and/or the region contour.

According to a fourth aspect of embodiments of the present disclosure, there is provided a data processing apparatus, including: an acquisition module, configured to acquire expression data and image data of a biological tissue sample, wherein the expression data is obtained by sectioning the biological tissue sample and performing spatiotemporal omics sequencing on a section, and the image data is obtained by acquiring an image of a sectioned biological tissue sample; a calculation module, configured to calculate the expression data to obtain an expression image; an equalization module, configured to perform a histogram equalization on the expression image and the image data to obtain an image pair of the expression image-the image data; a registration module, configured to perform a rigid registration and an elastic registration in sequence for the image pair to generate contour data of the biological tissue sample, wherein the contour data is used to generate the three-dimensional display data of the biological tissue sample.

According to a fifth aspect of embodiments of the present disclosure, there is provided an electronic device, including: at least one processor; and a memory communicatively connected to the at least one processor, wherein the memory stores instructions executable by the at least one processor, and when the instructions executed by the at least one processor, the at least one processor execute the method according to the first aspect or the second aspect of embodiments.

According to a sixth aspect of embodiments of the present disclosure, there is provided a non-transitory computer-readable storage medium including computer executable instructions, wherein when the computer executable instructions are executed by a processor, the method according to the first aspect or the second aspect of embodiments.

According to the embodiments of the present disclosure, the data acquisition method includes sectioning a biological tissue sample and performing spatiotemporal omics sequencing on a section to obtain expression data; acquiring an image of a sectioned biological tissue sample to obtain image data; registering the expression data and/or the image data to generate an external contour and/or a region contour of the biological tissue sample; generating a three-dimensional display data of the biological tissue sample based on the external contour and/or the region contour. The method according to the embodiments of the present disclosure, combining the in-situ captured spatiotemporal omics technology with optical imaging information, assisted by the rigid and elastic registration of serial tissue sections, enables three-dimensional reconstruction and obtains internal and external tissue contours for the positioning of visualization information, thereby achieving effective processing and display for large-scale and high-resolution spatiotemporal omics data.

Additional aspects and advantages of embodiments of the present disclosure will be given in part in the following descriptions, become apparent in part from the following descriptions, or be learned from the practice of the embodiments of the present disclosure.

### BRIEF DESCRIPTION OF THE DRAWINGS

The above and/or additional aspects and advantages of embodiments of the present disclosure will become apparent and more readily appreciated from the following descriptions made with reference to the drawings, in which:

FIG. 1 is a flow diagram of a data acquisition method according to an embodiment of the present disclosure;

FIG. 2 is a flow diagram of a data acquisition method according to an embodiment of the present disclosure;

FIG. 3 is a schematic diagram of a data acquisition method according to an embodiment of the present disclosure;

FIG. 4 is a schematic diagram showing a cryostat according to an embodiment of the present disclosure;

FIG. 5 is a schematic diagram illustrating a sliding position of a camera according to an embodiment of the present disclosure;

FIG. 6 is a schematic diagram of a method for preprocessing expression data according to an embodiment of the present disclosure;

FIG. 7 is a schematic diagram showing a rule of image acquisition and spatiotemporal sequencing according to an embodiment of the present disclosure;

FIG. 8 is a schematic diagram showing sample images acquired by an imaging device according to an embodiment of the present disclosure;

FIG. 9 is a schematic diagram showing a strategy of image segmentation processing according to an embodiment of the present disclosure;

FIG. 10 is a schematic diagram showing a strategy of image segmentation processing according to an embodiment of the present disclosure;

FIG. 11 is a schematic diagram showing a rigid registration algorithm according to an embodiment of the present disclosure;

FIG. 12 is a schematic diagram showing results of image segmentation and registration according to an embodiment of the present disclosure;

FIG. 13 is a flow diagram of generating an external contour of a tissue according to an embodiment of the present disclosure;

FIG. 14 is a schematic diagram showing a result of image rendering according to an embodiment of the present disclosure;

FIG. 15 is a schematic diagram showing a result of the image segmentation and the registration according to an embodiment of the present disclosure;

FIG. 16 is a flow diagram of registering the expression data according to an embodiment of the present disclosure;

FIG. 17 is a schematic diagram showing a result of a histogram equalization according to an embodiment of the present disclosure;

FIG. 18 is a flow diagram of a rigid registration according to an embodiment of the present disclosure;

FIG. 19 is a schematic diagram showing a result of the rigid registration according to an embodiment of the present disclosure;

FIG. 20 is a flow diagram of an elastic registration according to an embodiment of the present disclosure;

FIG. 21 is a schematic diagram showing a result of the elastic registration according to an embodiment of the present disclosure;

FIG. 22 is a flow diagram of performing serial registrations on the expression data according to an embodiment of the present disclosure;

FIG. 23 is a flow diagram of generating a region contour model according to an embodiment of the present disclosure;

FIG. 24 is a schematic diagram showing a basic data structure of a master file according to an embodiment of the present disclosure;

FIG. 25 is a schematic diagram showing a data structure of single-gene based hierarchical and block data in the master file according to an embodiment of the present disclosure;

FIG. 26 is a schematic diagram showing a data structure of cell-type based hierarchical and block data in the master file according to an embodiment of the present disclosure;

FIG. 27 is a schematic diagram of a result of mouse brain reconstruction and rendering according to an embodiment of the present disclosure;

FIG. 28 is a schematic diagram showing a generation result of an external contour model for a tissue according to an embodiment of the present disclosure;

FIG. 29 is a schematic diagram showing a generation result of a region contour for a tissue according to an embodiment of the present disclosure;

FIG. 30 is a schematic diagram showing a displayable single-gene list of the mouse brain according to an embodiment of the present disclosure;

FIG. 31 is a schematic diagram showing a data processing method according to an embodiment of the present disclosure;

FIG. 32 is a schematic block diagram showing a data acquisition apparatus according to an embodiment of the present disclosure;

FIG. 33 is a schematic block diagram showing a data processing apparatus according to an embodiment of the present disclosure;

FIG. 34 is a structural schematic diagram showing an electronic device according to an embodiment of the present disclosure.

### DETAILED DESCRIPTION

Embodiments of the present disclosure will be described in detail and examples of embodiments are illustrated in the drawings. The same or similar elements and the elements having the same or similar functions are denoted by like reference numerals throughout the descriptions. Embodiments described herein with reference to drawings are explanatory, serve to explain the present disclosure, and are not construed to limit embodiments of the present disclosure.

Since the development of spatiotemporal omics technology, it has been preliminarily applied in biology fields such as neuroscience, pathology, and developmental biology. During the in-situ capture-based method, in-situ spatial information and various omics information are typically obtained by physical contact between microchips and tissue sections as well as probe capture. However, the method is limited to application to only two-dimensional (2D) planar spatiotemporal omics analysis of tissues due to the destructive sectioning of tissue and the planar capture of relevant information, making it difficult to obtain three-dimensional morphological structures and accurate positional information. Given that different organs and tissues have unique three-dimensional cell distribution and gene expression profiles, it is of great technical challenge and significant scientific importance to conduct three-dimensional spatiotemporal omics researches on tissues.

The key to three-dimensional spatiotemporal omics lies in how to overcome the limitations of 2D plane research, and achieve comprehensive analysis of the spatial distribution of different cells, cell-cell interactions, expression of genes and proteins, and overall morphological structure at the three-dimensional level.

The current three-dimensional reconstruction workflow of spatiotemporal omics mainly involves performing rigid registration between adjacent or proximate sections, then generating a region contour based on the clustering or annotation information, and finally generating the overall contour of the entire tissue/organism based on the section stack. The results of three-dimensional reconstruction are usually presented in the form of figures in articles or demo images in code repositories. This approach has the deficiencies as follows:
(1) The current three-dimensional reconstruction of spatiotemporal omics performs rigid registration on the expression data measured on the 2D plane, which only relies on the abundance information of expression captured by spatiotemporal omics. The deformation of expression data caused by manual operations and biochemical processes during experiments are not corrected, thus failing to reconstruct the data back to the in-situ expression level;
(2) The rigid registration based on the abundance information of expression requires that the abundance information of expression captured from adjacent or proximate sections. If the sections are far apart, the accuracy of rigid registration will be reduced;
(3) The restored three-dimensional spatial information needs to be projected onto 2D planes such as computer screens and paper journal articles for visualization. Without other auxiliary information (e.g., boundary lines and surfaces), the three-dimensional effect provided by scattered points in 2D planes is limited. This is particularly unfavorable for human eyes to identify three-dimensional spatial information when the number of scattered points is large and there is no obvious tissue contour information;
(4) Due to limitations of technology and samples, the number of information points on a single tissue section is limited. When the physical size of the sample and microchip resolution increase, downstream analysis and display are restricted by hardware conditions, resulting in insufficient capability to process large-volume tissue sample data and conduct post-processing display.

Therefore, a data acquisition method, apparatus, and system are proposed in embodiments of the present disclosure. The method combines the in-situ captured spatiotemporal omics technology with optical imaging information, to obtain original tissue contour for assisting the positioning of internally captured biological information. This enables the restoration of three-dimensional positional information of the tissue before sectioning, reduces the impact of experimental operations on tissue morphology, achieves position registration at the cellular resolution for each information collection site on the chip, and effective processing and display for large-size and large-data volume tissue samples.

The data acquisition scheme provided in the present disclosure will be described in detail below with reference to the drawings.

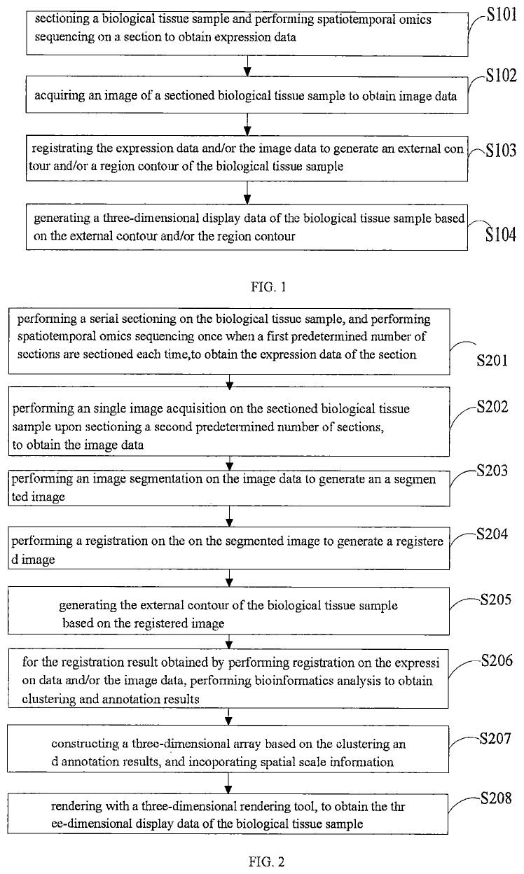

FIG. 1 is a flow diagram of a data acquisition method according to an embodiment of the present disclosure. As shown in FIG. 1, the method may include the following steps.

In the step S101, a biological tissue sample is sectioned, and spatiotemporal omics sequencing is performed on the section, to obtain expression data.

Specifically, serial sectioning is performed on the biological tissue sample, and the section at a designated position is selected for spatiotemporal omics sequencing according to actual conditions.

In an embodiment of the present disclosure, the biological tissue sample is derived from a biological specimen, typically referring to body fluid (such as blood, urine, saliva, bile, gastric juice, lymph, and other secretions of organisms), hair, muscle, and certain tissues or organs (such as thymus, pancreas, liver, lung, brain, stomach, kidney, etc.) of animals (including human), as well as various microorganisms.

In an embodiment of the present disclosure, the equipment used for sectioning the biological tissue sample is not limited. Preferably, a biological tissue cryostat is used for sectioning, which provides higher precision and stability.

In an embodiment of the present disclosure, expression data is acquired based on an in-situ capture method, which may involve capturing signals from a sequencing chip. The performance of the chip influences the technical effects, such as precision and resolution. Preferably, in-situ capture for a tissue is performed using a DNA nanoball sequencing chip, which, due to its large size and high signal density, achieves superior resolution and field of view. Additionally, other spatiotemporal omics data based on in-situ capture sequencing (such as proteomics data currently under development) may also be applied, and the present disclosure is not limited thereto.

In an embodiment of the present disclosure, when selecting sections for spatiotemporal omics sequencing each time, the corresponding positions of the sections are recorded, and these positions are used for subsequent three-dimensional reconstruction.

In the step S102, an image of a sectioned biological tissue sample is acquired to obtain image data.

In an embodiment of the present disclosure, the image acquisition of a sectioned biological tissue sample may be carried out by optical microscopy, or by other in-situ imaging techniques that do not affect spatiotemporal omics, and the present disclosure is not limited thereto.

In an embodiment of the present disclosure, image acquisition is performed on the sectioned tissues corresponding to all selected sections subjected to spatiotemporal omics sequencing, mainly on the cross-section thereof for subsequent registration. The image acquisition may also be performed on other sectioned biological tissue sample, and the specific selection rules are not restricted by embodiments of the present disclosure. For example, the image acquisition is performed on the sectioned biological tissue sample once for every m cuts, and a slice obtained from every n cuts is subjected to spatial-temporal omics sequencing, where m and n are positive integers respectively and may be adjusted according to actual conditions, and n may be a multiple of m.

In an embodiment of the present disclosure, when selecting sections for image acquisition each time, the corresponding positions of the sections are recorded, and these positions are used for subsequent three-dimensional reconstruction.

In the step S103, a registration is performed on the expression data and/or the image data to generate an external contour and/or a region contour of the biological tissue sample.

Optionally, before registration, the method further includes performing preprocessing on the expression data and performing image segmentation on the image data. The preprocessing may include at least one of aggregation processing, quality control, logarithmic normalization, highly variable gene identification, or dimensionality reduction. During the image segmentation, tissue segmentation is performed using an image segmentation algorithm, so that unqualified segmented samples may be identified and removed, and qualified segmentation results are retained as image data for subsequent registration.

In an embodiment of the present disclosure, the method may include performing registration on the image data to generate the external contour of the biological tissue sample.

In an embodiment of the present disclosure, an image segmentation is performed on the image data to generate a segmented image; a registration is performed on the segmented image to generate a registered image; and the external contour of the biological tissue sample is generated based on the registered image finally. In an embodiment, the first segmented image is selected for rotation correction, and then the corrected image is used as the first reference image for registration of other images of segmentation. Specifically, rigid registration or other registration methods may be used, and the present disclosure is not limited thereto.

In an embodiment of the present disclosure, the method may include performing registration on the expression data and the image data to generate a region contour of the biological tissue sample.

In an embodiment of the present disclosure, the expression data is calculated to obtain an expression image; a histogram equalization is performed on the expression image and the image data to obtain an image pair of the expression image-the image data; a rigid registration and an elastic registration are performed in sequence for the image pair to generate the region contour.

In an embodiment of the present disclosure, when image data is not available (e.g., due to the image acquisition is not performed or other reasons), the method may further include performing registration on the expression data to generate the external contour and/or the region contour of the biological tissue sample, provided that the sections for spatiotemporal omics involved in registration are adjacent or proximate.

In an embodiment of the present disclosure, a registration is performed on expression data from two adjacent or proximate sections to obtain registered expression data; the external contour and/or the region contour of the biological tissue sample are generated based on the registered expression data.

It should be understood that the external contour and/or the region contour of the biological tissue sample may be generated by any one of the approaches described above, which may be selected according to actual research or application requirements, and the present disclosure is not limited thereto.

In an embodiment of the present disclosure, contour data of the biological tissue sample is generated based on the registered spatiotemporal omics data. Specifically, for the registration result obtained by performing registration on the expression data and/or the image data, bioinformatics analysis is performed to obtain clustering and annotation results; a three-dimensional array is constructed based on the clustering and annotation results and incorporated with spatial scale information; and each clustering or block result is segmented to generate smooth contour surfaces of multiple tissues or regions in the internal structure of the sample. In an embodiment, each clustering or block result may be segmented using tools such as the Segment Editor in 3D Slicer, and finally the generated contour images are stored in common 3D vector model data formats such as vtk, obj, or stl.

In the step S104, three-dimensional display data of the biological tissue sample is generated based on the external contour and/or the region contour.

In an embodiment of the present disclosure, three-dimensional display data of the biological tissue sample is obtained by rendering with a 3D rendering tool, and the rendering tool is not limited by the present disclosure.

In an embodiment of the present disclosure, the external contour and/or region contour data is integrated with other image rendering data and registered spatiotemporal omics data to generate a project master file. To balance transmission efficiency and rendering quality, the spatiotemporal omics data is subjected to hierarchical and block processing from perspectives such as single-gene expression levels and cell types to generate the project master file. The generated master file is loaded into a renderer through a flask-based back-end service, enabling intuitive display of gene expression distribution, tissue contours, and other information in three-dimensional space.

In summary, according to the data acquisition method provided by the embodiment of the present disclosure, a biological tissue sample is sectioned, and spatiotemporal omics sequencing is performed on the section, to obtain expression data; an image of a sectioned biological tissue sample is acquired to obtain image data; a registration is performed on the expression data and/or the image data to generate an external contour and/or a region contour of the biological tissue sample; and three-dimensional display data of the biological tissue sample is generated based on the external contour and/or the region contour. The method of the present disclosure combines in-situ capture spatiotemporal omics technology with optical imaging information for registration to obtain internal and external tissue contours, which assist in positioning visualization information. This enables effective processing and display for large-size, high-resolution spatiotemporal omics data, as well as enables simultaneous analysis of genes and images by the application in three-dimensional spatiotemporal omics technology.

FIG. 2 is a flow diagram of a data acquisition method according to an embodiment of the present disclosure. Based on the embodiment shown in FIG. 1, as shown in FIG. 2, the method may include the following steps. To facilitate understanding, FIG. 3 shows a schematic diagram of a data acquisition method according to an embodiment of the present disclosure.

In the present disclosure, to facilitate understanding of the solution, an embodiment, process, and effect of the method are further described in detail. Taking the data acquisition of rhesus monkey (Macaca mulatta) brain as an example, specific descriptions will be provided in the embodiments of each step. It should be understood that the example is not construed to limit embodiments of the present disclosure.

Based on the embodiment shown in FIG. 1, step S101 includes step S201 shown in FIG. 2.

In the step S201, serial sectioning is performed on the biological tissue sample, and spatiotemporal omics sequencing is performed upon sectioning a first predetermined number of sections, to obtain the expression data of the sections.

In an embodiment of the present disclosure, the first predetermined number is a positive integer. For example, when every 10 sections are sectioned, one is selected for spatiotemporal omics sequencing.

In an embodiment of the present disclosure, the biological tissue sample may preferably be sectioned with a biological tissue cryostat, which provides higher precision and stability. Specifically, the biological tissue sample is mounted on the cryostat head of the cryostat. According to the requirements for subsequent imaging, the position and angle of the cryostat head are roughly adjusted. Trimming is performed on the tissue sections at first to remove excess Optimal Cutting Temperature Compound (OCT) of embedding medium until the tissue region is exposed, and then serial sectioning is performed on the tissue.

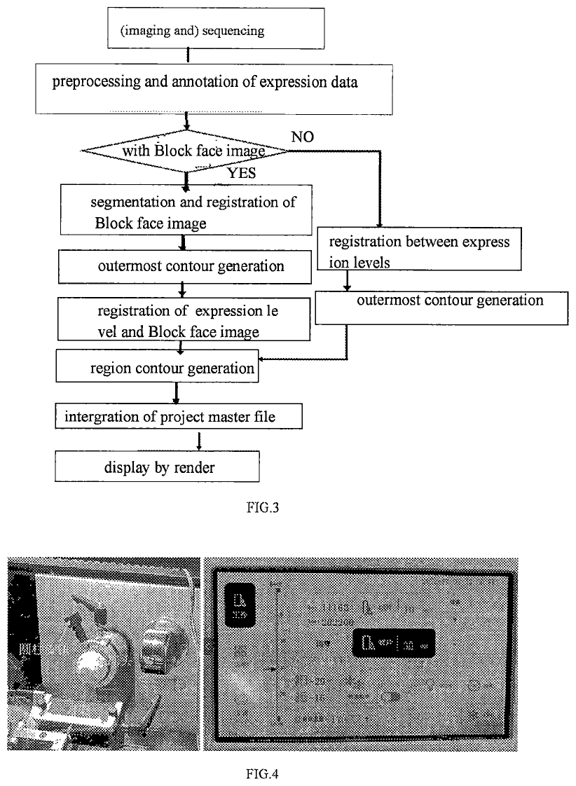

In an embodiment of the present disclosure, the data acquisition of rhesus monkey brain is taken as an example, which uses a cryostat for sectioning. As shown in FIG. 4, first, the rhesus monkey brain tissue sample is prepared and fixed at the center of the cryostat head of the cryostat by the experimenter. Second, the OCT (frozen section embedding medium) embedding block containing the monkey brain tissue to be sectioned is mounted onto the sample holder, and the section plane of the tissue is adjusted to an appropriate angle. The trimming mode is set on the display screen shown in FIG. 4, and set the trimming thickness. According to the sample embedding status information provided by the customer, the handwheel on the right is turned to perform a rough-trimming operation, so that the OCT block face is trimmed flat. Before focusing, ensure that the advance-scale pointer of the cryostat head indicates approximately 15 mm. Therefore, if the embedding block is estimated to jump out of this range, the cryostat head needs to be retracted. At this time, the retraction button should be pressed, and the retraction scale is determined according to the remaining thickness of the monkey brain tissue. Next, the knife holder needs to be adjusted to fit the tissue surface. Specifically, if the thickness of the embedding block is about 10 mm, there is no need to adjust the knife holder; if the monkey brain tissue is thicker than 10 mm (e.g., an embedding block with a thickness of 15 mm), the cryostat head must be retracted by at least 5 mm in thickness to compensate for its maximum forward travel of 10 mm. The specific retraction value may be read from the display screen on the right. At this time, the knife holder is advanced until its edge substantially abuts the tissue surface. The first cut at this stage is used for flatness adjustment and should be discarded, and a new round of experiments starts from the second cut.

In an embodiment of the present disclosure, expression data is acquired based on an in-situ capture method, which may involve capturing signals from a sequencing chip. The performance of the chip influences the technical effects, such as precision and resolution. Preferably, in-situ capture for a tissue is performed using a DNA nanoball sequencing chip, which, due to its large size and high signal density, achieves superior resolution and field of view. Additionally, other spatiotemporal omics data based on in-situ capture sequencing (such as proteomics data currently under development) may also be applied, and the present disclosure is not limited thereto.

In some embodiments of the present disclosure, after obtaining the expression data of the section, the method further includes performing preprocessing on the expression data. The preprocessing may include at least one of aggregation processing, quality control, logarithmic normalization, highly variable gene identification, or dimensionality reduction.

In the present disclosure, the data preprocessing reduces noise in the expression data and mitigate the data scale effect caused by experimental conditions and operations. This enables more accurate annotation results, the expression image that better reflect tissue structure, and more accurate display results in the renderer.

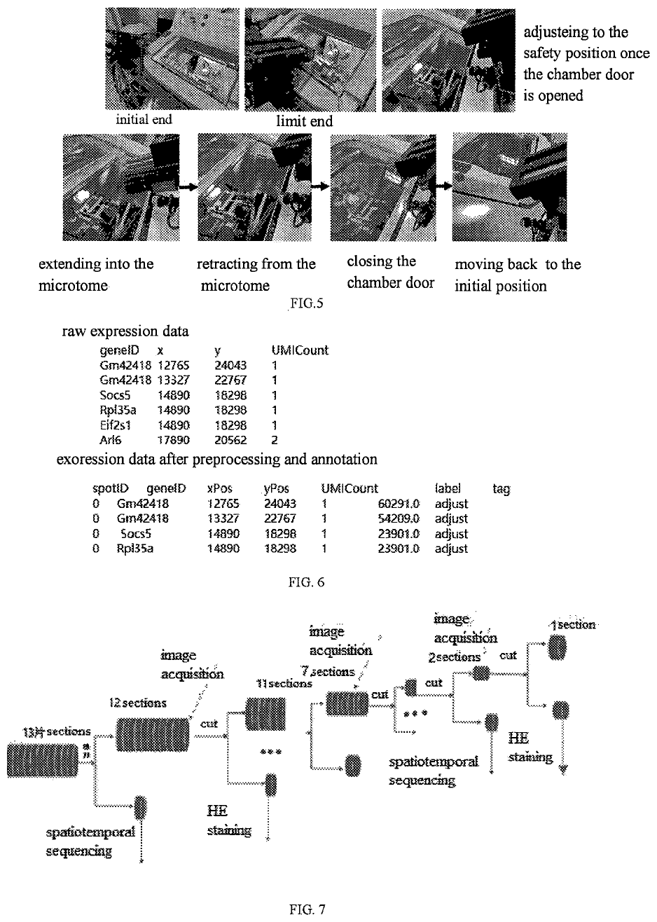

In the present disclosure, as shown in FIG. 6, the method further includes annotating the data. The annotation results based on preprocessing will be used for generating region contours and to drive visualization of the data in the renderer.

It should be noted that the specific preprocessing and annotation methods are determined by the user of the workflow. Existing workflows, such as workflows involving binset aggregation, quality control, logarithmic normalization (also referred to as “sequencing depth normalization”), highly variable gene identification, dimensionality reduction, and clustering, may be used, or self-designed workflows may be adopted.

In an embodiment of the present disclosure, the data acquisition of rhesus monkey brain is taken as an example, as shown in FIG. 6, the upper part shows the raw expression data of rhesus monkey brain section, and the lower part shows the corresponding expression data preprocessed and annotated.

Based on the embodiment shown in FIG. 1, step S102 includes step S202 shown in FIG. 2.

In the step S202, the single image acquisition is performed for the sectioned biological tissue sample upon sectioning a second predetermined number of sections, to obtain the image data. where the second predetermined number is a positive integer.

In an embodiment, the second predetermined number is a positive integer.

In an embodiment of the present disclosure, the sequencing and the image acquisition are performed according to specific rules, where the first predetermined number is a multiple of the second predetermined number. Spatiotemporal omics sequencing is necessarily accompanied by imaging, while imaging may sometimes be performed without sequencing. Adjustments are made according to actual conditions. For example, as shown in FIG. 7, the first predetermined number is 10 and the second predetermined number is 5. The biological tissue sample is sectioned into 13 sections. After the first section is collected, spatiotemporal omics sequencing is performed on said first section and image acquisition is performed on the sectioned biological sample. After further 5 sections are obtained by the serial sectioning, image acquisition is performed on the sectioned biological sample. After another 5 sections are obtained by the serial sectioning, spatiotemporal omics sequencing is performed on the section and the image acquisition is performed on the sectioned biological sample, until the sectioning ends.

In an embodiment of the present disclosure, the image acquisition of a sectioned biological tissue sample may be carried out by optical microscopy, or by other in-situ imaging techniques that do not affect spatiotemporal omics, and the present disclosure is not limited thereto.

In an embodiment of the present disclosure, the image acquisition of a sectioned biological tissue sample may be performed using an imaging device. Specifically, as shown in FIG. 5, the imaging device is aligned with the tissue section, and system calibration is performed to bring the sample cross-section into focus. The current position of a camera is recorded through the sliding table software control panel. The tissue is sectioned according to the plan, and the cutter sequence is recorded. Following the collection of the first section for spatiotemporal omics sequencing, the remaining tissue block face is subjected to BlockFace imaging. The specific rules for imaging and spatiotemporal omics sequencing are as shown in FIG. 7.

In an embodiment of the present disclosure, the data acquisition of rhesus monkey brain is taken as an example. The image acquisition of a sectioned biological tissue sample may be performed using in-situ optical imaging with the imaging device as shown in FIG. 4. First, the imaging device is connected to a computer. Specifically, a suitable lens is selected and adjusted so that the scale value aligned with the white dot on indicates the actual magnification. The camera, the power cable of sliding table and USB signal cable are connected respectively, where the USB signal cable is connected to the USB 3.0 interface of the computer. The camera imaging software is opened on the computer desktop, and the camera “MV-CH250-90UC” is selected to ensure normal connection of the camera. Second, the sliding table software and camera software are operated to control the imaging device for image acquisition. Specifically, the sliding table software is launched, and set to “Easy Mode”. Then, the servo is then enabled, causing the sliding table operation interface appears. A travel distance (e.g., 100 mm, typically 80-100 mm) of the sliding table is entered for both forward and reverse directions, and the exact magnitude being determined with reference to the initial focal plane. When the imaging device has not moved to the imaging position of the cryostat, only the value is set without activating “+”. As shown in FIG. 5, the imaging device is slid from the initial end to the limit end along the slide rail, and then adjusted to the safety position once the chamber door is opened. The “Video” button of the camera software is selected, and a real-time video display will appear in the video display window of the camera. Then, the “+” button of the sliding table, with the input value 100 as the setting aforementioned, is clicked to control the sliding table to extend into the cryostat. The tissue cross-section will then appear in the display frame. If the tissue surface is not in the focal plane, the sliding unit may be set, and the unit “1” can be entered in the input frame of the sliding table software. The “−” and “+” operations are selected according to actual conditions, where selecting “−” means the sliding table extends outward, and selecting “+” means the sliding table extends inward. Through this fine adjustment, the black Mark points formed on the planed OCT surface are brought into sharp focus within the camera frame. Before marking the Mark, it is necessary to ensure that the OCT has been cut flat by the cryostat. With the OCT block confirmed to lie in the focal plane and the alignment marks in focus, serial sections are advanced until the target tissue surface is just revealed, whereupon the regular sectioning protocol is initiated. At this time, formal sectioning may be performed according to the sectioning workflow. Finally, the image is captured and saved, and the imaging device is reset. Specifically, for the BlockFace image that need to be captured and saved, the “Capture Image” button on the camera imaging window is clicked to save the current image. After the single imaging operation is completed, the operator clicks “origin reset” button in the sliding table software, causing the imaging device to be automatically retracted from the chamber and the chamber door to close (FIG. 5). At this time, the experimenter moves the imaging device back to the initial position, and a single imaging operation is completed. The imaging result is as shown in FIG. 8.

Steps S203 to S205 shown in FIG. 2 are further disclosures of step S103, performing registration on the expression data and/or the image data to generate an external contour and/or a region contour of the biological tissue sample, in the embodiment of FIG. 1.

In the step, S203, the image segmentation is performed on the image data to generate a segmented image.

In an embodiment of the present disclosure, specifically, the BlockFace images captured by optical imaging are subjected to tissue segmentation using an image segmentation algorithm, so that unqualified segmented samples may be identified and removed, and qualified segmentation results are retained as image data for subsequent processing.

In some embodiments of the present disclosure, the step of performing image segmentation on the image data includes: converting the image data to HSV color space, extracting and converting image data of H-channel to a grayscale image; binarizing the image data using a preset image processing algorithm to determine a binary contour image; retaining a contour image meeting a preset condition and merging the retained contour image with the image data.

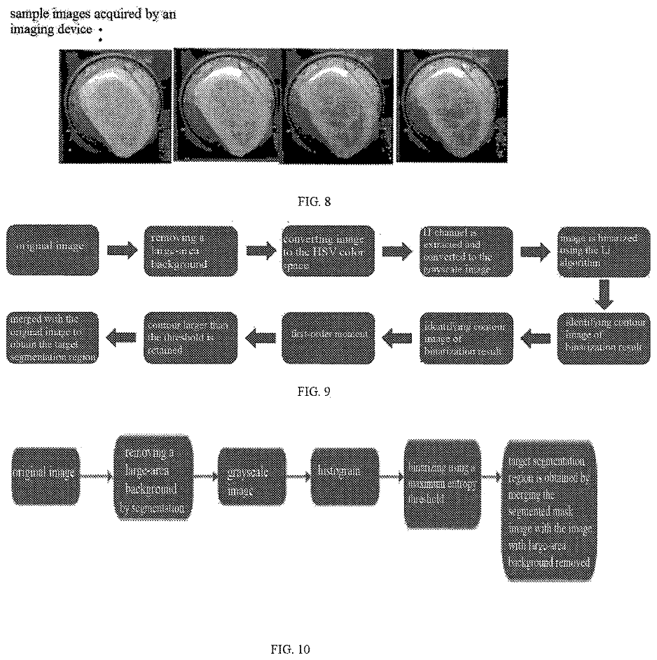

Specifically, as shown in FIG. 9, the BlockFace original image is segmented to remove a large-area background first. The image is converted to the HSV color space format, and the H-channel is extracted and converted to the grayscale image. The image may be binarized using the Li algorithm, and the c binary contour image is identified. Through the first-order moment of the contour, contour larger than the threshold is retained and merged with the original image to obtain the target segmentation region.

Optionally, the step of performing image segmentation on the image data may further include: obtaining a grayscale image and a histogram of the image data; binarizing the image data using a maximum entropy threshold; merging the binary image with the image data.

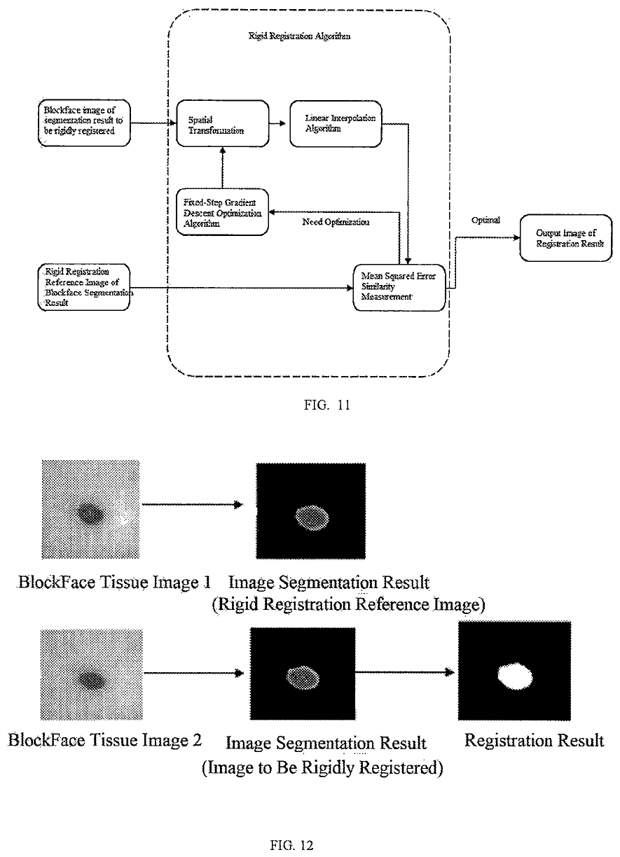

In the present disclosure, the image segmentation may be performed preferably using the segmentation strategy shown in FIG. 9. For images that are not suitable for this strategy or are incorrectly segmented by this strategy, the segmentation strategy shown in FIG. 10 may be optionally used. For example, due to the diversities of BlockFace images of tissue regions, if the pixel values of the background and the target tissue to be segmented in the BlockFace image are very close, the threshold obtained by the Li algorithm may not be applied to all cases.

Specifically, as shown in FIG. 10, the BlockFace original image is segmented to remove a large-area background first. Then, the image with the large-area background removed is converted to the grayscale image, and the histogram of the grayscale image is statistically analyzed, which means a grayscale histogram the frequencies of all pixels in a digital image are counted according to their grayscale values. The maximum entropy binarization threshold is obtained by maximizing the information measure between the target and the background, and this threshold is used for segmentation of grayscale image. The target segmentation region is obtained by merging the segmented mask image with the image with large-area background removed.

In the step S204, a registration is performed on the segmented image to generate a registered image.

In an embodiment of the present disclosure, before the registration, the segmented image of the first BlockFace image is selected for rotation correction, and then the corrected image is used as the first reference image for registration of other images of segmentation.

In some embodiments of the present disclosure, the step of registering the segmented image to generate a registered image includes: performing a processing of a spatial transformation and a linear interpolation algorithm on the segmented image; measuring a mean squared error similarity between the segmented image after processing and a reference image of registration; determining an optimal measurement solution as the registered image.

In an embodiment of the present disclosure, the logic of registering the BlockFace image of segmentation result is as shown in FIG. 11. For the fixed-step gradient descent optimization algorithm for the rigid registration algorithm, the maximum number of iterations is set to K, the learning rate is set to r, and the step size is set to t (values of K, r, and t are adjusted according to actual conditions), thereby enables the registration. Specifically, the processing of the spatial transformation and the linear interpolation algorithm are performed on the BlockFace image of segmentation result first. the segmented image of the first BlockFace image is used as the reference image. The mean squared error similarit metric between the transformed image and the reference image of registration is performed. If the metric has not converged, the gradient-descent update with fixed step size r is executed, followed by the processing of the spatial transformation and the linear interpolation algorithms again. This process is repeated until the maximum number of iterations K is reached, whereupon the optimal result is output as the registered image.

For steps S203 and S204, taking the BlockFace images of mouse heart tissue as an example, the BlockFace image segmentation result and registration result of mouse heart tissue are shown in FIG. 12.

In the step S205, the external contour of the biological tissue sample is generated based on the registered image.

In some embodiments of the present disclosure, the step of generating the external contour of the biological tissue sample based on the registered image includes: stacking all registered images into an array file with a preset format; processing the array file to obtain the external contour of the biological tissue sample.

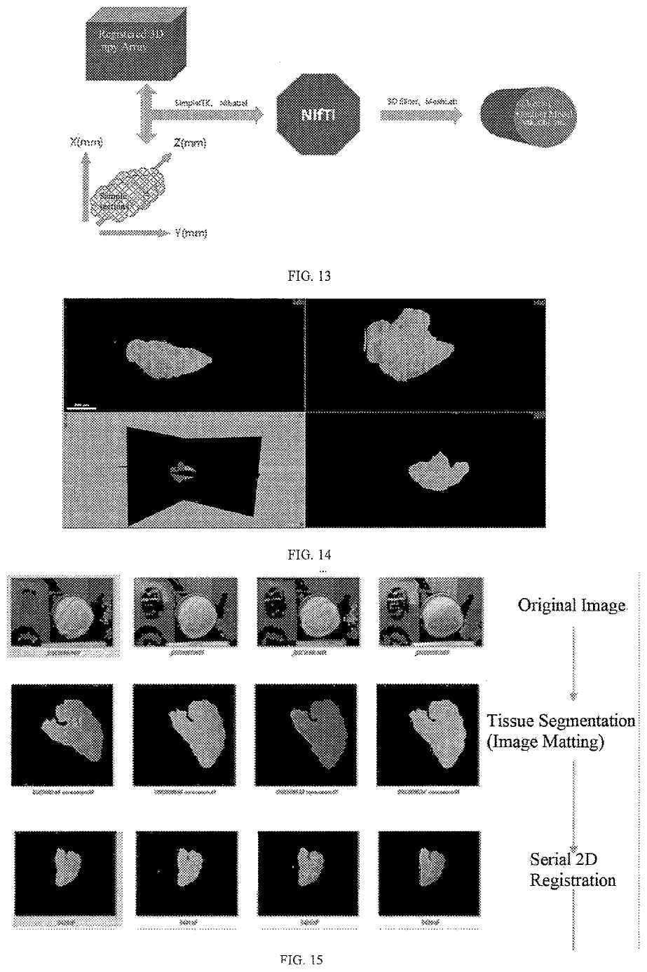

In an embodiment of the present disclosure, as shown in FIG. 13, Volume and Mesh generation are performed on the registered images. Specifically, the registered BlockFace segmentation images are stacked into a single file in .npy array. in which non-zero voxels correspond to tissue and zero voxels to background. Therefore, the file may be stored in the medical image format, such as Neuroimaging Informatics Technology Initiative (NIfTI). Using image processing tools such as SimpleITK and Nibabel, supplemented by the three-dimensional spatial scale of the sections, the file is stored in .nii format. Then, using tools such as 3D Slicer, the “segment-make surface” operation is performed to form a smooth external tissue contour, which is stored in common 3D vector model data formats such as vtk, obj, or stl.

In an embodiment of the present disclosure, the method further includes processing the registered optical imaging results into a format required for rendering.

Specifically, the registered BlockFace images of segmentation are stacked into a file in .npy or .tif format. Using Python toolkits such as CloudVolume and Igneous, the BlockFace images of segmentation are processed into binary rendering files with a hierarchical portioned structure supported by the renderer. The final rendering result is shown in FIG. 14.

In an embodiment of the present disclosure, the data acquisition of rhesus monkey brain is taken as an example. As shown in FIG. 15, the BlockFace captured images may be subjected to rotation correction using ImageJ, and the Region of Interest (ROI) analysis region selection. Then, segmentation of the target tissue object and background is performed on the BlockFace ROI images, and the BlockFace images of segmentation are registered to form a BlockFace image stack spatially aligned. The outermost contour of the object based on the BlockFace segmentation images is generated using the algorithm.

In some embodiments of the present disclosure, after the step of generating the external contour of the biological tissue sample in steps S203 to S205, the method further includes: calculating the expression data to obtain an expression image; performing a histogram equalization on the expression image and the image data to obtain an image pair of the expression image-the image data; performing a rigid registration and an elastic registration in sequence for the image pair to generate the region contour.

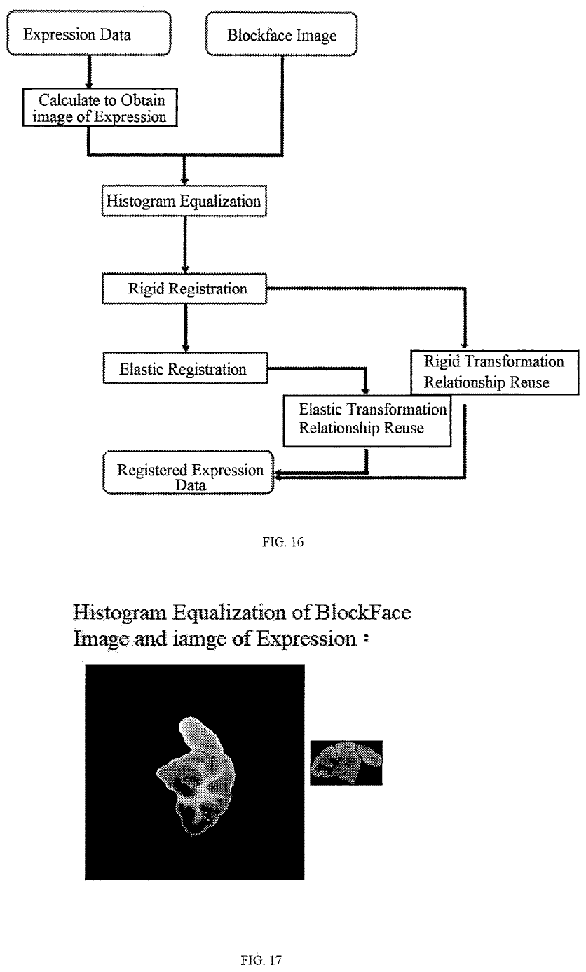

Specifically, as shown in FIG. 16, the spatiotemporal omics expression data is processed as a spatial abundance map, which reflects the abundance of expression levels across different regions. Histogram equalization is performed on the expression image and the BlockFace image of segmentation respectively. The result of histogram equalization is shown in FIG. 17. Then for the image pair of the BlockFace image of segmentation-the expression image, both are in adjacent section positions, the operations are performed as follows. The rigid registration and the elastic registration between the BlockFace image of segmentation serving as the reference image and the expression image is performed. When the two types of registration are completed, the transformation relationships determined during the registrations are applied to the expression data, thereby achieving the registration between the expression data and the BlockFace image of segmentation. In an embodiment, the transformation relationship of the rigid registration is in the form of a 3×3 transformation matrix, and the transformation relationship of the elastic-registration transformation consists of two deformation fields that respectively encode the x-and y-direction displacement distributions.

Optionally, the step of performing a rigid registration for the image pair includes: downsampling the image pair respectively; extracting feature information from the image pair; performing a similarity-estimated affine transformation on the image pair to obtain a rigid registration result.

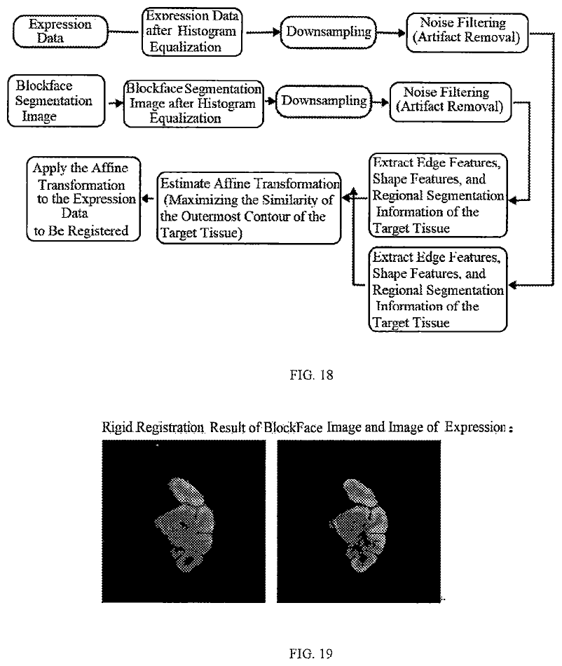

In an embodiment of the present disclosure, as shown in FIG. 18, the downsampling and the noise filtering for artifacts removal is performed on the image pair, respectively. The edge features, shape feature, and region segmentation information of the target tissue in the both images of different modalities are extracted through a deep learning model, respectively. Then, the similarity-estimated affine transformation is performed at the model output by maximizing the similarities of the outermost contour of the target tissue. Finally, the affine transformation is applied to the original image of the expression data to be registered. The pair of processed expression data and the processed BlockFace image of segmentation result are transmitted for the elastic registration processing. The image of the rigid registration result is shown in FIG. 19.

Optionally, the step of performing an elastic registration on the image pair includes: performing a histogram equalization on the rigid registration result; inputting the rigid registration result into a deep learning model to output an intermediate result by the deep learning model, where the intermediate result is a deformation field, where the deep learning model is trained using the image pair of the expression image-the image data; performing nearest-neighbor interpolation transformation on the rigid registration result based on the deformation field to obtain an elastic registration result.

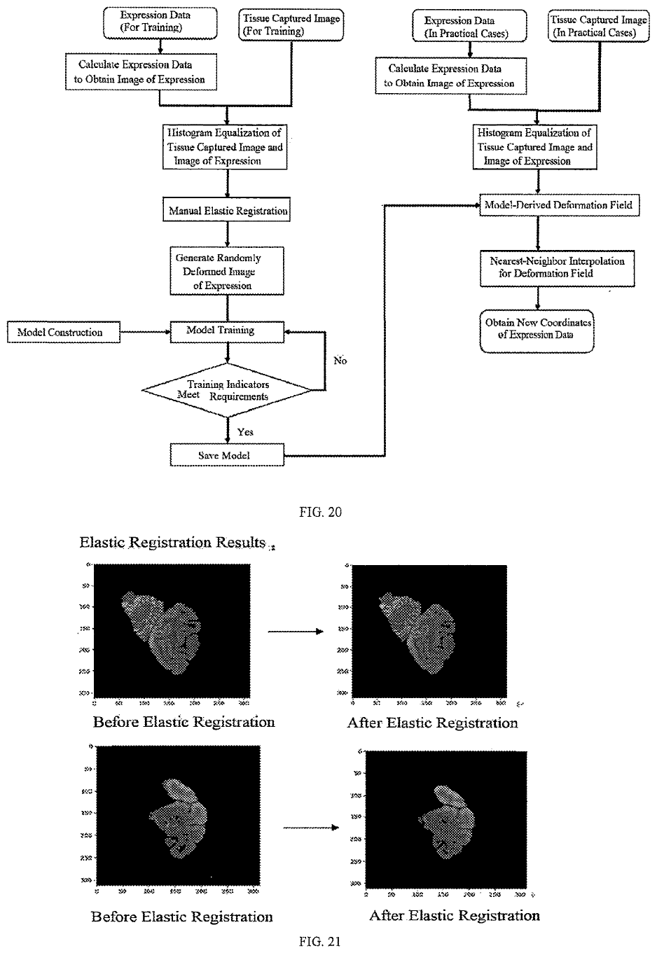

In an embodiment of the present disclosure, the elastic registration is enabled based on the derivation of the deep learning model, as shown in FIG. 20. The corresponding deep learning model is obtained by the following steps. The expression data is processed into the expression image first, then the histogram equalization is performed on the expression image and the corresponding BlockFace image of segmentation, respectively. Then, the rigid registration is performed on the image pair of the expression image-BlockFace image of segmentation in software as the input image for model training, and the elastic registration is further performed to obtain elastic registration results as the label for model training. Based on the control point coordinates output during manual elastic registration by the software, random parameters are added to generate randomly deformed expression image, so that data synthesis is achieved. Finally, supervised models are trained using the synthesized data and real data, and the training is stopped when the training indicators meet the requirements, and the model is saved. The deep learning model is obtained and may be used to perform a derivation of elastic deformation. The expression data and the BlockFace image of segmentation in practical cases are processed according to the above methods to obtain the histogram-equalized images. The two images are input into the model, and the intermediate result of the model, i.e. the deformation field, is output. Based on the deformation field, the corresponding displacement is obtained to perform nearest-neighbor interpolation transformation on the expression data, so that new expression data is calculated. As shown in FIG. 21, taking the monkey brain as an example, the transformation of the correspondence between the expression image-BlockFace image before and after the elastic registration is shown, where the red one is the BlockFace image, and the green one is the expression image. After the elastic registration, the visible deformation of the expression image is corrected.

In another optional embodiment, the expression data may also be directly used for registration to generate the external contour and/or region contour of the biological tissue sample. The method includes: performing the registration on the expression data from two adjacent or proximate sections to obtain registered expression data; generating the external contour and/or the region contour of the biological tissue sample based on the registered expression data.

It should be understood that in the case of no image acquisition, for example, the imaging is not performed due to user habits, lack of imaging device, etc., the method may still generate the external contour and/or the region contour of the biological tissue sample, provided that the sections for spatiotemporal omics involved in registration are adjacent or proximate.

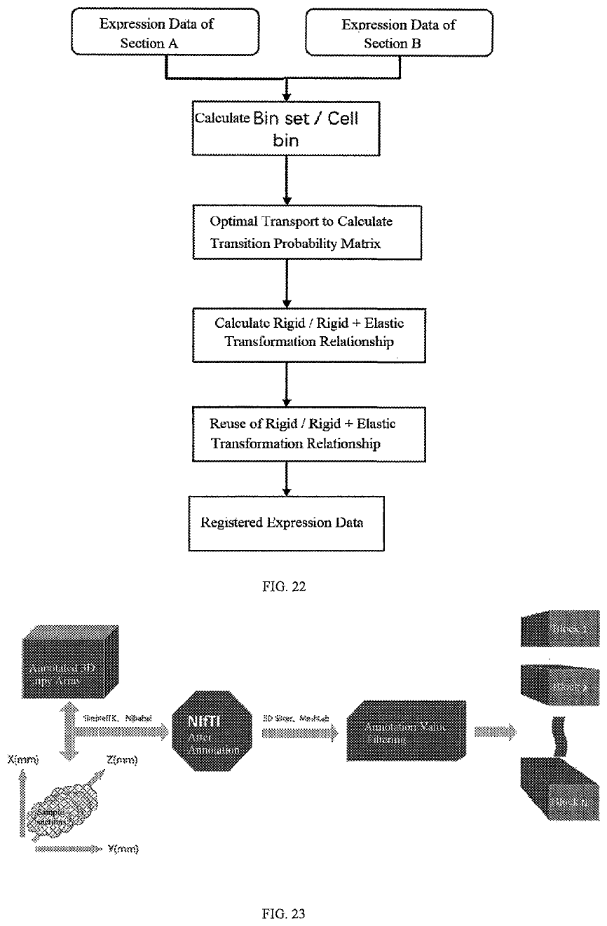

In an embodiment of the present disclosure, as shown in FIG. 22, a bin set or cell bin is calculated based on the expression data from two adjacent or proximate sections. After preprocessing such as logarithmic normalization, the optimal transport problem is solved to obtain the transition probability matrix of the optimal transport, which is also referred to as the pi matrix for short. After performing post-processing on the pi matrix, rigid or rigid+elastic transformation relationships may be calculated. Finally, this transformation relationship is applied to the raw expression data.

In the step S206, for the registration result obtained by performing registration on the expression data and/or the image data, bioinformatics analysis is performed to obtain clustering and annotation results.

In the step S207, a three-dimensional array is constructed based on the clustering and annotation results and incorporated with spatial scale information

In an embodiment of the present disclosure, the external contour and/or the region contour model is generated based on the registration result obtained by performing registration on the expression data and/or the image data.

In an embodiment of the present disclosure, the external contour is generated based on the registration result obtained by performing registration on the image data, specific details are as shown in FIG. 13, with reference to step S205.

In an embodiment of the present disclosure, the region contour is generated based on the registration result obtained by performing registration on the expression data and/or the image data, specifically, similar to step S205. As shown in FIG. 23, for the registered expression data, conventional bioinformatics analysis is performed on each bin set or cell bin to obtain the clustering and annotation results. A 3D .npy array is constructed using the annotation or clustering label of each bin set or cell bin, and spatial scale information is incorporated to obtain a NIfTI file based on the annotation and clustering results. Each clustering or region result may be segmented using tools such as the Segment Editor in 3D Slicer to generate a smooth contour surface of multiple tissues or regions in the internal structure of the sample, and the generated contour are stored in common 3D vector model data formats such as vtk, obj, or stl. This corresponds to the step S205 above.

In an embodiment of the present disclosure, the generated external contour and/or region contour model is processed into a binary 3D model file bound to region labels through the Python toolkit multiresolution-mesh-creator.

In an embodiment of the present disclosure, the image rendering data, registered spatiotemporal omics data, tissue contour data, region contour data, and the like described above are integrated into a project master file, and the content structure of the file depends on research or application requirements.

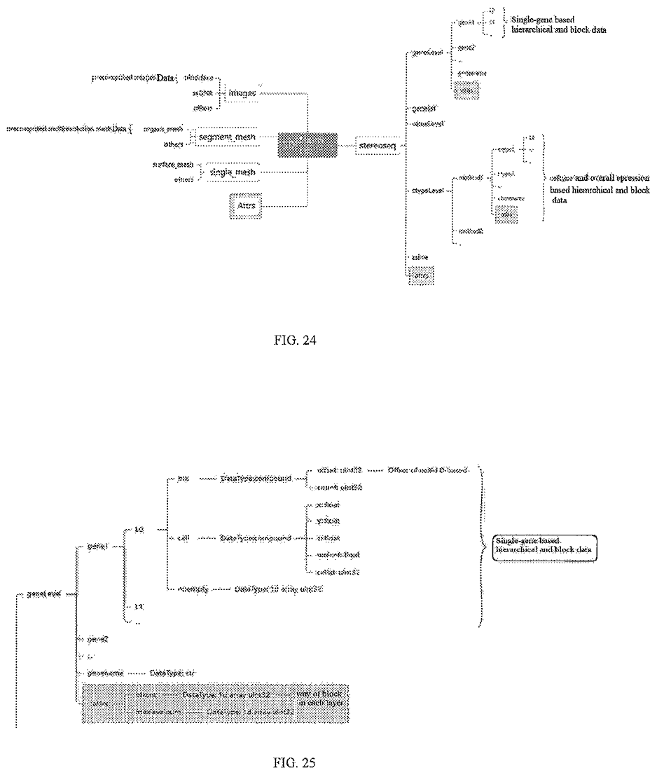

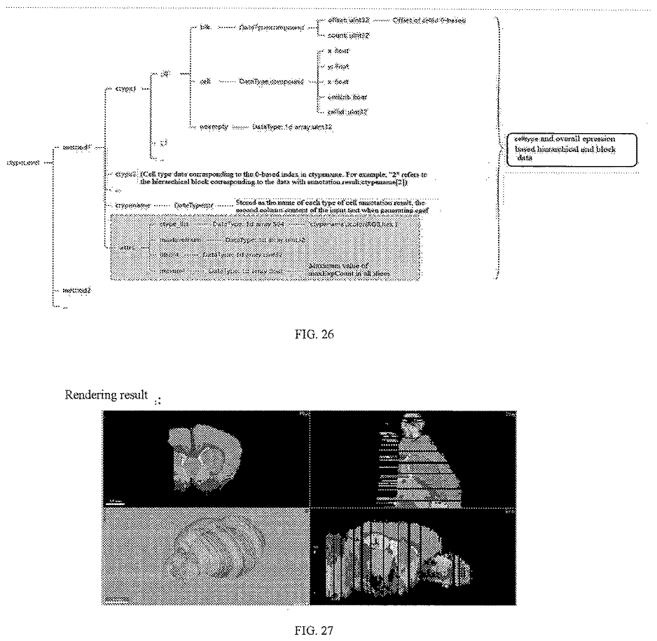

In an embodiment of the present disclosure, the registered spatiotemporal omics expression data is converted into a cgef format file through geftools, and then combined with the binary image data, binary region contour model data, tissue external contour model data generated in the steps described above to generate a master file through gef3d. The content structure of the file is shown in FIG. 24, mainly showing the top three hierarchical levels of the master file.

In some embodiments of the present disclosure, c to balance transmission efficiency and rendering effects, the spatiotemporal omics data is subjected to hierarchical and block processing from perspectives such as single-gene expression levels and cell types, when generating the project master file.

In an embodiment of the present disclosure, hierarchical and block processing refers to performing sampling in a pyramid-like structure on the a large number of data point clouds according to spatial positions, ensuring that the union set of data points in all blocks of all layers constitute the entire data. This processing is automatically performed when gef3d generates the master file, and the threshold for the number of bins/cell bins per block needs to be specified.

In an embodiment of the present disclosure, the hierarchical and block processing method may include: first, traversing all cgefs to obtain the overall spatial size and setting a maximum number of layers; calculating the division scale from the respective spatial extents of the data points along the three orthogonal axes; and pre-dividing the number of blocks in each layer from top to bottom according to an 8-section method, with each block being approximately a cube. By default, there is only one block in the first layer. Through the first traversal, data of each gene in each section including coordinates and expression level abundance, namely the attribute values of data points in the entire space, may be obtained. Then, performing hierarchical and block processing on the data of single gene; based on the results calculated in the first step, from the top layer to the bottom layer, filling each small block of each layer with data from the overall data of gene in sequence, until the small block is full (this is controlled by the threshold); go on filling the next layer with the remaining part, where if the data volume of any block in the next layer is too small (this is also controlled by the threshold), the data of this block will be added to the parent block of the previous layer. The data allocation proceeds downward in sequence until the gene data is fully allocated. The hierarchical and block processing of cell type annotation results, such as celltype, is similar to that of single genes.

The storage structure of the expression level or cell type data after hierarchical and block processing in the master file is shown in FIGS. 25 and 26. Under the LO directory, “blk” represents block, which is an abbreviation of block. This directory is used to store block data information. Under the blk directory, “offset” represents the offset, storing the starting index offset of the block data, and “count” represents the number, storing the number of points of the block data; “cell” represents the cell, this “cell” directory is used to store all bin/cellbin data-related information under this layer. Under the cell directory, x, y, and z store the x, y, and z coordinate values of the bin/cellbin spatial coordinates respectively, “umicnt” stores the bin/cellbin expression level, and “cellid” represents the cell serial numbers, storing the bin/cellbin serial number. Under the attrs directory, “ctype_list” represents the cell type list, storing the list of cell type serial numbers, names, and colors; “maxlevelnum” represents the maximum number of layers, which is the maximum number of layers among all cell type data; “blkcnt” represents the number of blocks, storing the number of blocks in each axis direction of different layers; “maxumi” represents the maximum expression level, storing the maximum value of the expression level.

In the step S208, three-dimensional display data of the biological tissue sample is obtained by rendering with a three-dimensional rendering tool.

Specifically, the generated master file through the above steps is loaded into a renderer through a flask-based back-end service, enabling intuitive display of gene expression distribution, tissue contours, and other information in three-dimensional space. The display on the web side is shown in FIG. 27, where the overall expression data and the tissue external contour model are loaded respectively.

In summary, the data acquisition method provided by the embodiment of the present disclosure combines in-situ capture spatiotemporal omics technology with optical imaging information, assisted by the rigid and elastic registration of serial tissue sections, enables three-dimensional reconstruction and obtains internal and external tissue contours for the positioning of visualization information, thereby achieving effective processing and display for large-scale and high-resolution spatiotemporal omics data.

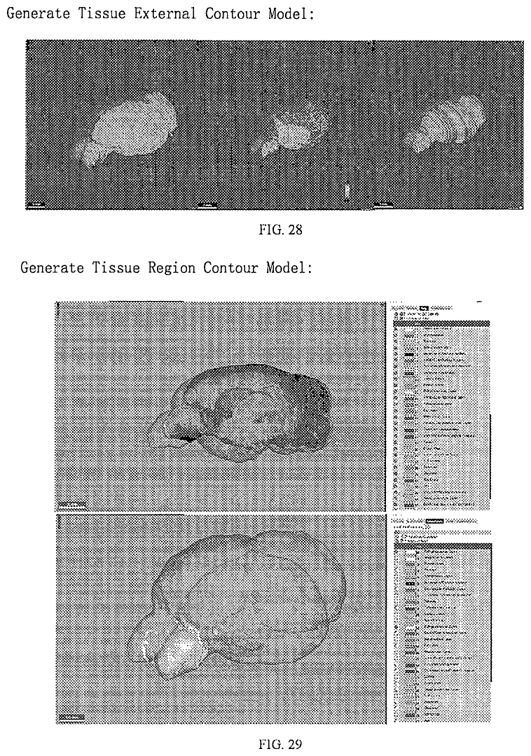

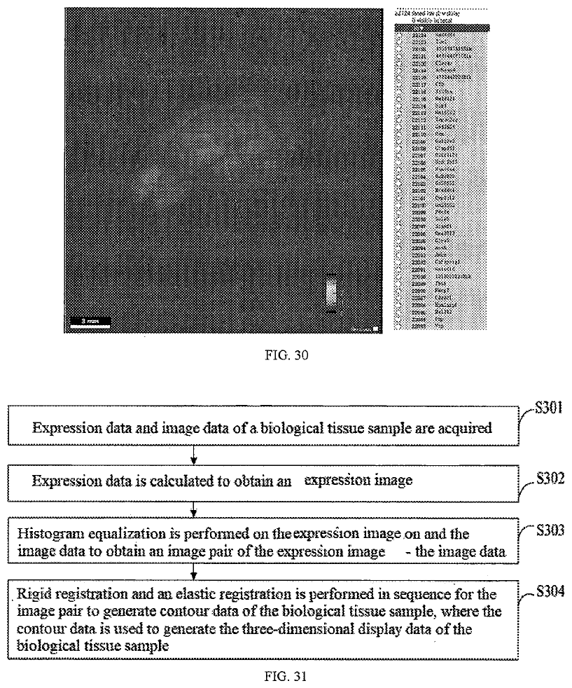

The effect of the method of the present disclosure is shown as follows:
1. Taking the BlockFace images of mouse heart tissue as an example, the segmentation and registration result for the BlockFace images of mouse heart tissue are shown in FIG. 12, where image segmentation processing and image registration are performed on the collected sample image data through steps S204 and S205.
2. Taking the monkey brain as an example, according to the method of FIG. 18, which shows the transformation of the correspondence between the expression image and collected image before and after the elastic registration, the result is shown in FIG. 19, where after the elastic registration, the visible deformation of the expression image is corrected.
3. A tissue external contour model is generated and displayed in combination with spatial scattered point information such as transcriptome, as shown in FIG. 28. The display of mouse brain tissue contour and cell type, display of mouse brain tissue contour and single-gene expression level, and display of mouse brain tissue contour model and total gene expression level are shown from left to right.
4. A tissue region contour model is generated, as shown in FIG. 29. The display of the mouse brain tissue region contour model is shown on the left side, and the display of the mouse brain tissue region contour model and the corresponding cell type bins are shown on the right side.
5. The processing and display of large-size and large-volume tissue sample are shown in FIG. 30. In the example, the mouse brain data of more than 100 spatiotemporal omics chips is displayed. The chip size is 1 cm×1 cm, and the displayed data includes about 900K coordinate points, covering approximately 22K genes.

Therefore, the method of the present disclosure achieves better restoration of the tissue contour and related expression data of the tissue sample before sectioning. Applied to spatiotemporal omics three-dimensional reconstruction, The method can help promote the research and application of three-dimensional spatiotemporal omics technology.

FIG. 31 is a flow diagram showing a data processing method according to an embodiment of the present disclosure. As shown in FIG. 31, the method may include the following steps.

In the step S301, expression data and image data of a biological tissue sample are acquired.

Where, the expression data is obtained by sectioning the biological tissue sample and performing spatiotemporal omics sequencing on a section, and the image data is obtained by acquiring an image of a sectioned biological tissue sample.

In an embodiment of the present disclosure, serial sectioning is performed on the biological tissue sample, and section at designated position is selected for spatiotemporal omics sequencing according to actual conditions to acquire expression data.

In an embodiment of the present disclosure, the image acquisition of a sectioned biological tissue sample may be carried out by optical microscopy, or by other in-situ imaging techniques that do not affect spatiotemporal omics, and the present disclosure is not limited thereto.

In an embodiment of the present disclosure, the biological tissue sample is derived from a biological specimen, typically referring to body fluid (such as blood, urine, saliva, bile, gastric juice, lymph, and other secretions of organisms), hair, muscle, and certain tissues or organs (such as thymus, pancreas, liver, lung, brain, stomach, kidney, etc.) of animals (including human), as well as various microorganisms.

In an embodiment of the present disclosure, the equipment used for sectioning the biological tissue sample is not limited. Preferably, a biological tissue cryostat is used for sectioning, which provides higher precision and stability.

In an embodiment of the present disclosure, expression data is acquired based on an in-situ capture method, which may involve capturing signals from a sequencing chip. The performance of the chip influences the technical effects, such as precision and resolution. Preferably, in-situ capture for a tissue is performed using a DNA nanoball sequencing chip, which, due to its large size and high signal density, achieves superior resolution and field of view. Additionally, other spatiotemporal omics data based on in-situ capture sequencing (such as proteomics data currently under development) may also be applied, and the present disclosure is not limited thereto.

In an embodiment of the present disclosure, image acquisition is performed on the sectioned tissues corresponding to all selected sections subjected to spatiotemporal omics sequencing, mainly on the cross-section thereof for subsequent registration. The image acquisition may also be performed on other sectioned biological tissue sample, and the specific selection rules are not restricted by embodiments of the present disclosure.

In an embodiment of the present disclosure, when selecting sections for spatiotemporal omics sequencing or image acquisition each time, the corresponding positions of the sections are recorded, and these positions are used for subsequent three-dimensional reconstruction.

In some embodiments of the present disclosure, after obtaining the expression data, the method further includes performing preprocessing on the expression data. The preprocessing may include at least one of aggregation processing, quality control, logarithmic normalization, highly variable gene identification, or dimensionality reduction. The data preprocessing helps reduce noise in the expression data and mitigate the data scale effect caused by experimental conditions and operations. This enables more accurate annotation results, the expression image that better reflect tissue structure, and more accurate display results in the renderer.

In some embodiments of the present disclosure, after acquiring the image data, the method may further include performing segmentation processing on the image data. Specifically, the image data is converted to the HSV color space, the image data of the H-channel is extracted and converted to a grayscale image, the image data is binarized using a preset image processing algorithm to determine a binary contour image, and the contour image meeting a preset condition is retained and merged with the image data.

Reference may be made to the specific embodiments of step S201 and step S202, which are not repeated herein.

In the step S302, the expression data is calculated to obtain an expression image.

In an embodiment of the present disclosure, according to actual research demands, the correlation coefficient of the spatiotemporal omics expression data is calculated to obtain an expression image that reflect the expression abundance in different regions. The specific calculation, statistics, and mapping methods are not limited by the present disclosure.

In the step S303, a histogram equalization is performed on the expression image and the image data to obtain an image pair of the expression image-the image data.

In an embodiment of the present disclosure, histogram equalization is a simple and effective image enhancement technique. It changes the grayscale of respective pixels in the image by modifying the histogram of an image, and is mainly used to enhance the contrast of images with a small dynamic range. Due to the possible concentration of grayscale distribution in a narrow interval, the original image may not be sufficiently clear. For example, an overexposed image has grayscale levels concentrated in the high-brightness range, while underexposure concentrates the image's grayscale levels in the low-brightness range. Histogram equalization can transform the histogram of the original image into a uniformly distributed (equalized) form, which increases the dynamic range of grayscale differences between pixels, thereby achieving the effect of enhancing the overall contrast of the image.

In an embodiment of the present disclosure, through step S301, a BlockFace image is collected as image data, and segmentation processing is performed on the image to obtain a BlockFace image of segmentation. The image pair of the BlockFace image of segmentation-the expression image with adjacent section positions are selected as the image pair of the expression image-the image data.

In the step S304, a rigid registration and an elastic registration is performed in sequence for the image pair to generate contour data of the biological tissue sample, where the contour data is used to generate the three-dimensional display data of the biological tissue sample.

In an embodiment of the present disclosure, as shown in FIG. 16, the rigid registration and the elastic registration between the BlockFace image of segmentation serving as the reference image and the expression image is performed. When the two types of registration are completed, the transformation relationships determined during the registrations are reused on the expression data, thereby achieving the registration between the expression data and the BlockFace image of segmentation. In an embodiment, the transformation relationship of the rigid registration is in the form of a 3×3 transformation matrix, and the transformation relationship of the elastic registration is in the form of two deformation fields, representing the deformation distribution in the x and y directions respectively.

In an optional embodiment of the present disclosure, the step of performing a rigid registration for the image pair includes: downsampling the image pair respectively; extracting feature information from the image pair; performing a similarity-estimated affine transformation on the image pair to obtain a rigid registration result.

In an embodiment of the present disclosure, as shown in FIG. 18, the downsampling and the noise filtering for artifacts removal is performed on the image pair, respectively. The edge features, shape feature, and region segmentation information of the target tissue in the both images of different modalities are extracted through a deep learning model, respectively. Then, the similarity-estimated affine transformation is performed at the model output by maximizing the similarities of the outermost contour of the target tissue. Finally, the affine transformation is applied to the original image of the expression data to be registered. The pair of processed expression data and the processed BlockFace image of segmentation result are transmitted for the elastic registration processing. The image of the rigid registration result is shown in FIG. 19.

In an optional embodiment of the present disclosure, the step of performing an elastic registration on the image pair includes: performing a histogram equalization on the rigid registration result; inputting the rigid registration result into a deep learning model to output an intermediate result by the deep learning model, where the intermediate result is a deformation field, where the deep learning model is trained using the image pair of the expression image-the image data; performing nearest-neighbor interpolation transformation on the rigid registration result based on the deformation field to obtain an elastic registration result.

In an embodiment of the present disclosure, the elastic registration is enabled based on the derivation of the deep learning model, as shown in FIG. 20. The corresponding deep learning model is obtained by the following steps. The expression data is processed into the expression image first, then the histogram equalization is performed on the expression image and the corresponding BlockFace image of segmentation, respectively. Then, the rigid registration is performed on the image pair of the expression image-BlockFace image of segmentation in software as the input image for model training, and the elastic registration is further performed as the label for model training. Based on the control point coordinates output during manual elastic registration by the software, random parameters are added to generate randomly deformed expression image, so that data synthesis is achieved. Finally, supervised models are trained using the synthesized data and real data, and the training is stopped when the training indicators meet the requirements, and the model is saved. The deep learning model is obtained and may be used to perform a derivation of elastic deformation. The expression data and the BlockFace image of segmentation in practical cases are processed according to the above methods to obtain the histogram-equalized images. The two images are input into the model, and the intermediate result of the model, i.e. the deformation field, is output. Based on the deformation field, the corresponding displacement is obtained to perform nearest-neighbor interpolation transformation on the expression data, so that new expression data is calculated. As shown in FIG. 21, taking the monkey brain as an example, the transformation of the correspondence between the expression image-BlockFace image before and after the elastic registration is shown, where the red one is the BlockFace image, and the green one is the expression image. After the elastic registration, the visible deformation of the expression image is corrected.

In an embodiment of the present disclosure, contour data of the biological tissue sample is generated based on the registered spatiotemporal omics data. Specifically, based on the registered spatiotemporal expression, data conventional bioinformatics analysis is performed on each bin set or cell bin to obtain the clustering and annotation results. With the annotation or clustering result value of each bin set or cell bin as a unit, a three-dimensional .npy array is constructed, and spatial scale information is incorporated to obtain a NIfTI file based on the annotation and clustering results. Each clustering or block result may be segmented using tools such as the Segment Editor in 3D Slicer to generate a smooth contour surface of multiple tissues or regions in the internal structure of the sample, and the generated contour are stored in common 3D vector model data formats such as vtk, obj, or stl.

In the present disclosure, the contour data is used to generate three-dimensional display data of the biological tissue sample. Specifically, the contour data is integrated with other image rendering data, registered spatiotemporal omics data, and the like, to generate a project master file. Considering transmission efficiency and rendering effects comprehensively, when generating the project master file, the spatiotemporal omics data is subjected to hierarchical and block processing from perspectives such as single-gene expression levels and cell types. The generated master file is loaded into a renderer through a flask-based back-end service, enabling intuitive display of gene expression distribution, tissue contours, and other information in three-dimensional space.

Reference may be made to the specific embodiments corresponding to steps S203 to S208 in FIG. 2, which are not repeated herein.

In summary, according to the data processing method provided by the embodiment of the present disclosure, expression data and image data of a biological tissue sample are acquired, where the expression data is obtained by sectioning the biological tissue sample and performing spatiotemporal omics sequencing on a section, and the image data is obtained by acquiring an image of a sectioned biological tissue sample; the expression data is calculated to obtain an expression image; a histogram equalization is performed on the expression image and the image data to obtain an image pair of the expression image-the image data; a rigid registration and an elastic registration is performed in sequence for the image pair to generate contour data of the biological tissue sample, where the contour data is used to generate the three-dimensional display data of the biological tissue sample. The method provides a registration method for spatiotemporal sequencing expression data and image data of tissue section. By applying the present method, internal and external tissue contours are obtained for assisting the positioning of visualization information, which enables effective processing and display of large-size, high-resolution spatiotemporal omics data, and promotes the research and application of three-dimensional spatiotemporal omics technology.

To implement each function in the foregoing method provided in the embodiments of the present application, network device and the terminal device may include a hardware structure and a software module, and the foregoing functions may be implemented in a form of a hardware structure, a software module, or a combination of a hardware structure and a software module. A specific function among the foregoing functions may be performed by the hardware structure, the software module, or the combination of a hardware structure and a software module.

Corresponding to the data acquisition method provided by the several embodiments above, the present disclosure further provides a data acquisition apparatus. Since the data acquisition apparatus provided by the embodiment of the present disclosure corresponds to the data acquisition method provided by the several embodiments above, the specific embodiments of the data acquisition method are also applicable to the data acquisition apparatus provided by the embodiment, which will not be described in detail herein.

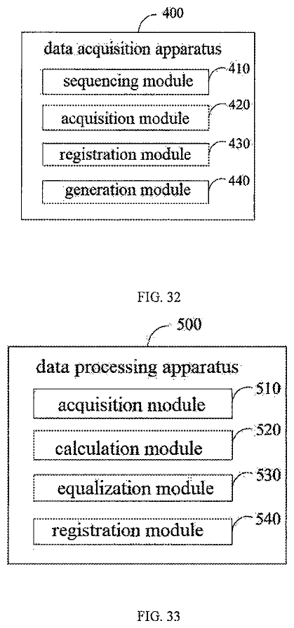

FIG. 32 is a schematic diagram showing a structure of a data acquisition apparatus 400 provided by an embodiment of the present disclosure. As shown in FIG. 32, the apparatus 400 may include: a sequencing module 410, configured to section a biological tissue sample and perform spatiotemporal omics sequencing on a section to obtain expression data; an acquisition module 420, configured to acquire an image of a sectioned biological tissue sample to obtain image data; a registration module 430, configured to perform a registration on the expression data and/or the image data to generate an external contour and/or region contour of the biological tissue sample; a generation module 440, configured to generate three-dimensional display data of the biological tissue sample based on the external contour and/or the region contour.

According to the data acquisition apparatus provided by the embodiment of the present disclosure, a biological tissue sample is sectioned, and spatiotemporal omics sequencing is performed on the section, to obtain expression data; an image of a sectioned biological tissue sample is acquired to obtain image data; a registration is performed on the expression data and/or the image data to generate an external contour and/or a region contour of the biological tissue sample; and three-dimensional display data of the biological tissue sample is generated based on the external contour and/or the region contour. The apparatus of the present disclosure combines in-situ capture spatiotemporal omics technology with optical imaging information for registration to obtain internal and external tissue contours, which assist in positioning visualization information. This enables effective processing and display for large-size, high-resolution spatiotemporal omics data.

In some embodiments, the sequencing module 410 is specifically configured to perform serial sectioning on the biological tissue sample, and perform spatiotemporal omics sequencing upon sectioning a first predetermined number of sections, to obtain the expression data of the sections, where the first predetermined number is a positive integer.

In some embodiments, the apparatus further includes a preprocessing module, configured to perform preprocessing on the expression data, where the preprocessing includes at least one of aggregation processing, quality control, logarithmic normalization, highly variable gene identification, or dimensionality reduction.

In some embodiments, the acquisition module 420 is specifically configured to perform a single image acquisition on the sectioned biological tissue sample upon sectioning a second predetermined number of sections, to obtain the image data, where the second predetermined number is a positive integer.

In some embodiments, the registration module 430 is specifically configured to: perform an image segmentation on the image data to generate a segmented image; perform a registration on the segmented image to generate a registered image; generate the external contour of the biological tissue sample based on the registered image.

In some embodiments, the step of performing an image segmentation on the image data includes: converting the image data to HSV color space, extracting and converting image data of H-channel to a grayscale image; binarizing the image data using a preset image processing algorithm to determine a binary contour image; retaining a contour image meeting a preset condition and merging the retained contour image with the image data.

In some embodiments, the method further includes: obtaining a grayscale image and a histogram of the image data; binarizing the image data using a maximum entropy threshold; merging the binary image with the image data.

In some embodiments, the step of registering the segmented image to generate a registered image includes: performing a processing of a spatial transformation and a linear interpolation algorithm on the segmented image; measuring a mean squared error similarity between the segmented image after processing and a reference image of registration; determining an optimal measurement solution as the registered image.

In some embodiments, the step of generating the external contour of the biological tissue sample based on the registered image includes: stacking all registered images into an array file with a preset format; processing the array file to obtain the external contour of the biological tissue sample.

In some embodiments, the registration module 430 is further configured to the expression data to obtain an expression image; perform a histogram equalization on the expression image and the image data to obtain an image pair of the expression image-the image data; perform a rigid registration and an elastic registration in sequence for the image pair to generate the region contour.

In some embodiments, the step of performing a rigid registration for the image pair includes: downsampling the image pair respectively; extracting feature information from the image pair; performing a similarity-estimated affine transformation on the image pair to obtain a rigid registration result.

In some embodiments, the step of performing an elastic registration on the image pair includes: performing a histogram equalization on the rigid registration result; inputting the rigid registration result into a deep learning model to output an intermediate result by the deep learning model, where the intermediate result is a deformation field, where the deep learning model is trained using the image pair of the expression image-the image data; performing nearest-neighbor interpolation transformation on the rigid registration result based on the deformation field to obtain an elastic registration result.

In some embodiments, the registration module 430 is further configured to: perform a registration on expression data from two adjacent or proximate sections to obtain registered expression data; generate the external contour and/or the region contour of the biological tissue sample based on the registered expression data.

In some embodiments, the generation module 440 is specifically configured to: for the registration result obtained by performing registration on the expression data and/or the image data, perform bioinformatics analysis to obtain clustering and annotation results; construct a three-dimensional array based on the clustering and annotation results, and incorporate spatial scale information; generate the three-dimensional display data of the biological tissue sample by rendering with a three-dimensional rendering tool.

In summary, according to the data acquisition method provided by the embodiment of the present disclosure, a biological tissue sample is sectioned, and spatiotemporal omics sequencing is performed on the section, to obtain expression data; an image of a sectioned biological tissue sample is acquired to obtain image data; a registration is performed on the expression data and/or the image data to generate an external contour and/or a region contour of the biological tissue sample; and three-dimensional display data of the biological tissue sample is generated based on the external contour and/or the region contour. The method combines in-situ capture spatiotemporal omics technology with optical imaging information, assisted by the rigid and elastic registration of serial tissue sections, enables three-dimensional reconstruction and obtains internal and external tissue contours for the positioning of visualization information, thereby achieving effective processing and display for large-scale and high-resolution spatiotemporal omics data, as well as enableing simultaneous analysis of genes and images by the application in three-dimensional spatiotemporal omics technology.

Corresponding to the data processing method provided by the several embodiments above, the present disclosure further provides a data processing apparatus. Since the data processing apparatus provided by the embodiment of the present disclosure corresponds to the data processing method provided by the several embodiments above, the specific embodiments of the data processing methods are also applicable to the data processing apparatus provided by the embodiment, which will not be described in detail herein.

FIG. 33 is a schematic diagram showing a structure of a data processing apparatus 500 provided by an embodiment of the present disclosure. As shown in FIG. 33, the apparatus 500 may include: an acquisition module 510, configured to acquire expression data and image data of a biological tissue sample, where the expression data is obtained by sectioning the biological tissue sample and performing spatiotemporal omics sequencing on a section, and the image data is obtained by acquiring an image of a sectioned biological tissue sample; a calculation module 520, configured to calculate the expression data to obtain an expression image; an equalization module 530, configured to perform a histogram equalization on the expression image and the image data to obtain an image pair of the expression image-the image data; a registration module 540, configured to perform a rigid registration and an elastic registration in sequence for the image pair to generate contour data of the biological tissue sample, where the contour data is used to generate the three-dimensional display data of the biological tissue sample.

According to the data processing apparatus provided by the embodiment of the present disclosure, expression data and image data of a biological tissue sample are acquired, where the expression data is obtained by sectioning the biological tissue sample and performing spatiotemporal omics sequencing on a section, and the image data is obtained by acquiring an image of a sectioned biological tissue sample; the expression data is calculated to obtain an expression image; a histogram equalization is performed on the expression image and the image data to obtain an image pair of the expression image-the image data; a rigid registration and an elastic registration is performed in sequence for the image pair to generate contour data of the biological tissue sample, where the contour data is used to generate the three-dimensional display data of the biological tissue sample. This achieves the registration of spatiotemporal sequencing expression data and image data of tissue section, and internal and external tissue contours are obtained for assisting the positioning of visualization information, which enables effective processing and display of large-size, high-resolution spatiotemporal omics data, and promotes the research and application of three-dimensional spatiotemporal omics technology.

In some embodiments, the registration module 540 is configured to perform a rigid registration on the image pair, which includes downsampling the image pair respectively; extracting feature information from the image pair; performing a similarity-estimated affine transformation on the image pair to obtain a rigid registration result.

In some embodiments, the registration module 540 is configured to perform an elastic registration on the image pair, which includes performing a histogram equalization on the rigid registration result; inputting the rigid registration result into a deep learning model to output an intermediate result by the deep learning model, where the intermediate result is a deformation field, where the deep learning model is trained using the image pair of the expression image-the image data; performing nearest-neighbor interpolation transformation on the rigid registration result based on the deformation field to obtain an elastic registration result.

In summary, according to the data processing method provided by the embodiment of the present disclosure, expression data and image data of a biological tissue sample are acquired, where the expression data is obtained by sectioning the biological tissue sample and performing spatiotemporal omics sequencing on a section, and the image data is obtained by acquiring an image of a sectioned biological tissue sample; the expression data is calculated to obtain an expression image; a histogram equalization is performed on the expression image and the image data to obtain an image pair of the expression image-the image data; a rigid registration and an elastic registration is performed in sequence for the image pair to generate contour data of the biological tissue sample, where the contour data is used to generate the three-dimensional display data of the biological tissue sample. The method provides a registration method for spatiotemporal sequencing expression data and image data of tissue section. By applying the present method, internal and external tissue contours are obtained for assisting the positioning of visualization information, which enables effective processing and display of large-size, high-resolution spatiotemporal omics data, and promotes the research and application of three-dimensional spatiotemporal omics technology.

In the embodiments provided by the present application above, the methods and apparatuses provided by the embodiments of the present application have been introduced. To implement each function in the foregoing method provided in the embodiments of the present application, an electronic device may include a hardware structure and a software module, and the foregoing functions may be implemented in a form of a hardware structure, a software module, or a combination of a hardware structure and a software module. A specific function among the foregoing functions may be performed by the hardware structure, the software module, or the combination of a hardware structure and a software module,

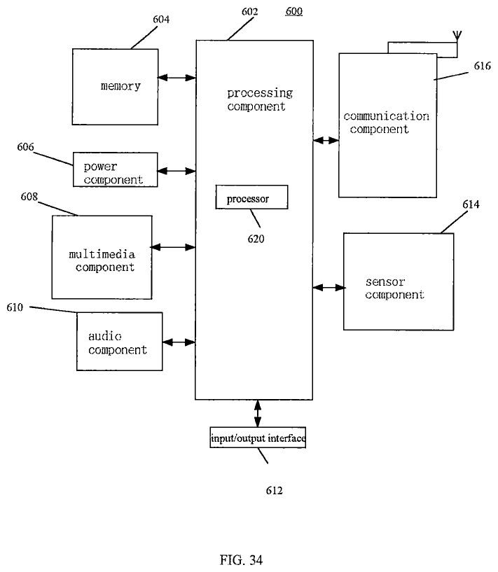

FIG. 34 is a block diagram of an electronic device 600 for implementing the above-described data acquisition method or data processing method according to an exemplary embodiment. For example, the electronic device 600 may be a mobile phone, a computer, a messaging device, a game console, a tablet device, a medical device, a fitness device, a personal digital assistant, or the like.

Referring to FIG. 34, the electronic device 600 may include one or more of the following components: a processing component 602, a memory 604, a power component 606, a multimedia component 608, an audio component 610, an input/output (I/O) interface 612, a sensor component 614, and a communication component 616.

The processing component 602 typically controls overall operations of the electronic device 600, such as operations associated with display, telephone calls, data communications, camera operations, and recording operations. The processing component 602 may include one or more processors 620 to execute instructions to perform all or part of the steps in the above-described methods. Moreover, the processing component 602 may include one or more modules to facilitate interaction between the processing component 602 and other components. For instance, the processing component 602 may include a multimedia module to facilitate interaction between the multimedia component 608 and the processing component 602.

The memory 604 is configured to store various types of data to support the operation of the electronic device 600. Examples of such data include instructions, contact data, phonebook data, messages, pictures, videos, and the like for any applications or methods operated on the electronic device 600. The memory 604 may be implemented using any type of volatile or non-volatile memory devices, or a combination thereof, such as a static random access memory (SRAM), an electrically erasable programmable read-only memory (EEPROM), an erasable programmable read-only memory (EPROM), a programmable read-only memory (PROM), a read-only memory (ROM), a magnetic memory, a flash memory, a magnetic disk, or an optical disk.

The power component 606 provides power to various components of the electronic device 600. The power component 606 may include a power management system, one or more power sources, and other components associated with the generation, management, and distribution of power in the electronic device 600.

The multimedia component 608 includes a screen that provides an output interface between the electronic device 600 and the user. In some embodiments, the screen may include a liquid crystal display (LCD) and a touch panel (TP). If the screen includes the touch panel, the screen may be implemented as a touch screen to receive input signals from the user. The touch panel includes one or more touch sensors to sense touches, swipes, and gestures on the touch panel. The touch sensors may not only sense a boundary of a touch or swipe action but also detect a period of time and pressure associated with the touch or swipe action. In some embodiments, the multimedia component 608 includes a front camera and/or a rear camera. When the electronic device 600 is in an operation mode, such as a shooting mode or a video mode, the front camera and/or the rear camera may receive external multimedia data. Each of the front camera and the rear camera may be a fixed optical lens system or have a focal length and optical zoom capability.

The audio component 610 is configured to output and/or input audio signals. For example, the audio component 610 includes a microphone (“MIC”) configured to receive an external audio signal when the electronic device 600 is in an operation mode, such as a call mode, a recording mode, and a voice recognition mode. The received audio signal may be further stored in the memory 604 or transmitted via the communication component 616. In some embodiments, the audio component 610 further includes a speaker to output audio signals.

The I/O interface 612 provides an interface between the processing component 602 and peripheral interface modules, such as a keyboard, a click wheel, buttons, and the like. The buttons may include, but are not limited to, a home button, a volume button, a power button, and a lock button.

The sensor component 614 includes one or more sensors to provide status assessments of various aspects of the electronic device 600. For instance, the sensor component 614 may detect an open/closed status of the electronic device 600, relative positioning of components, e.g., the display and the keypad of the electronic device 600, a change in position of the electronic device 600 or a component of the electronic device 600, a presence or absence of user contact with the electronic device 600, an orientation or acceleration/deceleration of the electronic device 600, and a change in temperature of the electronic device 600. The sensor component 614 may include a proximity sensor configured to detect the presence of nearby objects without any physical contact. The sensor component 614 may also include a light sensor, such as a CMOS or CCD image sensor, for use in imaging applications. In some embodiments, the sensor component 614 may further include an accelerometer sensor, a gyroscope sensor, a magnetic sensor, a pressure sensor, or a temperature sensor.

The communication component 616 is configured to facilitate communication, wired or wirelessly, between the electronic device 600 and other devices. The electronic device 600 can access a wireless network based on a communication standard, such as WiFi, 2G, 3G, 4G LTE, 5G New Radio (NR), or a combination thereof. In one exemplary embodiment, the communication component 616 receives a broadcast signal or broadcast-associated information from an external broadcast management system via a broadcast channel. In one exemplary embodiment, the communication component 616 further includes a near field communication (NFC) module to facilitate short-range communications. For example, the NFC module may be implemented based on radio frequency identification (RFID) technology, infrared data association (IrDA) technology, ultra-wideband (UWB) technology, Bluetooth (BT) technology, and other technologies.

In exemplary embodiments, the electronic device 600 may be implemented with one or more application specific integrated circuits (ASICs), digital signal processors (DSPs), digital signal processing devices (DSPDs), programmable logic devices (PLDs), field programmable gate arrays (FPGAs), controllers, microcontrollers, microprocessors, or other electronic components, for performing the above-described methods.

In exemplary embodiments, there is also provided a non-transitory computer-readable storage medium including instructions, such as the memory 604 including instructions, executable by the processor 620 of the electronic device 600 to perform the above-described methods. For example, the non-transitory computer-readable storage medium may be a ROM, a random access memory (RAM), a CD-ROM, a magnetic tape, a floppy disc, an optical data storage device, and the like.

Embodiments of the present disclosure further provide a non-transitory computer-readable storage medium including computer instructions, where the computer instructions are used for causing a computer to execute the data acquisition or data processing method described in the above embodiments of the present disclosure.

Embodiments of the present disclosure further provide a computer program product including a computer program, where when the computer program is executed by a processor, the data acquisition or data processing method described in the above embodiments of the present disclosure is executed.

Embodiments of the present disclosure further provide a chip, including one or more interface circuits and one or more processors; where the interface circuits are configured to receive a signal from a memory of an electronic device and send the signal to the processor, where the signal includes computer instructions stored in the memory; and the processor, when the computer instructions are executed by the processor, the electronic device executes the data acquisition or data processing method described in the above embodiments of the present disclosure.

It should be noted that the terms “first” and “second” in the specification, claims, and the above drawings of the present disclosure are used to distinguish similar objects, and are not necessarily used to describe a specific order or sequence. It should be understood that the data used in this way may be interchanged as appropriate, so that the embodiments of the present disclosure described herein may be implemented in an order other than those illustrated or described herein. The implementations described in the following illustrative embodiments do not represent all implementations consistent with the present disclosure. On the contrary, they are merely examples of apparatuses and methods consistent with some aspects of the present disclosure as detailed in the appended claims.

In the description of the present specification, references to the terms “an embodiment, “some embodiments”, “illustrative embodiments”, “examples”, “specific example”, “some examples” and the like, mean that a specific feature, structure, material, or characteristic described in connection with the implementation or example is included in at least one implementation or example of the present disclosure. In this specification, schematic representations of the above terms do not necessarily refer to the same implementation or example. Moreover, the described specific features, structures, materials, or characteristics may be combined in any suitable manner in any one or more implementations or examples.

Any process or method descriptions described in the flow diagram or otherwise herein may be understood as representing modules, segments, or portions of code including one or more executable instructions for implementing specific logical functions or processes. The scope of the preferred embodiments of the present disclosure includes additional implementations, where the functions may be executed not in the order shown or discussed, including in a substantially simultaneous manner or in the reverse order, depending on the functions involved, which should be understood by those skilled in the art to which the embodiments of the present disclosure belong.

The logic and/or steps represented in the flow diagram or otherwise described herein, for example, may be considered as a sequenced listing of executable instructions for implementing logical functions, and may be specifically implemented in any computer-readable medium for use by an instruction execution system, apparatus, or device (such as a computer-based system, a system including a processing module, or another system that can retrieve and execute instructions from the instruction execution system, apparatus, or device) or in combination with these instruction execution systems, apparatuses, or devices. For the purposes of this specification, a “computer-readable medium” may be any device that may contain, store, communicate, propagate, or transmit a program for use by or in combination with an instruction execution system, apparatus, or device. More specific examples (a non-exhaustive list) of the computer-readable medium include the following: an electrical connection having one or more wires (control methods), a portable computer diskette (magnetic device), a random access memory (RAM), a read-only memory (ROM), an erasable programmable read-only memory (EPROM or flash memory), an optical fiber device, and a portable compact disc read-only memory (CD-ROM). Additionally, the computer-readable medium may even be paper or another suitable medium on which the program can be printed, as the program can be obtained electronically by, for example, optically scanning the paper or other medium, then editing, interpreting, or otherwise processing it as necessary, and then storing it in computer memory.

It should be understood that each part of the embodiments of the present disclosure may be implemented by hardware, software, firmware, or a combination thereof. In the above embodiments, multiple steps or methods may be implemented by software or firmware stored in a memory and executed by a suitable instruction execution system. For example, if implemented by hardware, as in another embodiment, it may be implemented using any one or a combination of the following technologies known in the art: discrete logic circuits having logic gates for implementing logic functions on data signals, application-specific integrated circuits having appropriate combinational logic gates, programmable gate arrays (PGAs), field-programmable gate arrays (FPGAs), etc.

Those skilled in the art may understand that all or part of the steps carried by the methods of the above embodiments may be implemented by instructing relevant hardware through a program. The program may be stored in a computer-readable storage medium. When the program is executed, it includes one or a combination of the steps of the method embodiments.

In addition, each functional unit in each embodiment of the present disclosure may be integrated into one processing module, or each unit may exist physically independently, or two or more units may be integrated into one module. The above-described integrated modules may be implemented in the form of hardware or software functional modules. If the integrated modules are implemented in the form of software functional modules and sold or used as independent products, they may also be stored in a computer-readable storage medium. The storage medium mentioned above may be a read-only memory, a magnetic disk, an optical disk, or the like.

Although explanatory embodiments have been shown and described, it would be appreciated by those skilled in the art that the above embodiments cannot be construed to limit the present disclosure, and changes, alternatives, and modifications can be made in the embodiments in the scope of the present disclosure.

1. A data acquisition method, comprising:
sectioning a biological tissue sample and performing spatiotemporal omics sequencing on a section to obtain expression data;
acquiring an image of a sectioned biological tissue sample to obtain image data;
registering at least one of the expression data and the image data to generate at least one of an external contour and a region contour of the biological tissue sample;
generating a three-dimensional display data of the biological tissue sample based on at least one of the external contour and the region contour.

2. The method of claim 1, wherein said sectioning a biological tissue sample and performing spatiotemporal omics sequencing on a section to obtain expression data comprises:
performing serial sectioning on the biological tissue sample, and performing single spatiotemporal omics sequencing upon sectioning a first predetermined number of sections, to obtain the expression data of the sections,
wherein the first predetermined number is a positive integer.

3. The method of claim 2, further comprising:
performing preprocessing on the expression data,
wherein the preprocessing comprises at least one of aggregation processing, quality control, logarithmic normalization, highly variable gene identification, or dimensionality reduction.

4. The method according to claim 1, wherein said acquiring an image of the sectioned biological tissue sample to obtain image data comprises:
performing a single image acquisition on the sectioned biological tissue sample upon sectioning a second predetermined number of sections, to obtain the image data,
wherein the second predetermined number is a positive integer.

5. The method according to claim 1, wherein said performing registration on at least one of the expression data and the image data to generate at least one of an external contour and region contour of the biological tissue sample comprises:
performing an image segmentation on the image data to generate a segmented image;
registering the segmented image to generate a registered image;
generating the external contour of the biological tissue sample based on the registered image.

6. The method according to claim 5, wherein said performing an image segmentation on the image data comprises:
converting the image data to HSV color space and, extracting image data of H-channel;
converting the image data of H-channel to a grayscale image;
binarizing the image data using a preset image processing algorithm to determine a binary contour image;
retaining the binary contour image meeting a preset condition and merging the retained binary contour image with the image data.

7. The method according to claim 6, further comprising:
obtaining a grayscale image and obtaining a histogram of the image data based on the grayscale image;
binarizing the image data using a maximum entropy threshold;
merging the binary image with the image data.

8. The method according to claim 5, wherein said registering the segmented image to generate a registered image comprises:
performing a processing of a spatial transformation and a linear interpolation algorithm on the segmented image;
measuring a mean squared error similarity between the segmented image after processing and a reference image of registration;
determining an optimal measurement solution as the registered image.

9. The method according to claim 8, wherein said generating the external contour of the biological tissue sample based on the registered image comprises:
stacking all registered images into an array file with a preset format;
processing the array file to obtain the external contour of the biological tissue sample.

10. The method according to claim 5, wherein said performing registration on at least one of the expression data and the image data to generate an external contour and/or region contour of the biological tissue sample comprises:
calculating the expression data to obtain an expression image;
performing a histogram equalization on the expression image and the image data to obtain an image pair of the expression image-the image data;
performing a rigid registration and an elastic registration in sequence for the image pair to generate the region contour.

11. The method according to claim 10, wherein said performing a rigid registration for the image pair comprises:
downsampling the image pair respectively;
extracting feature information from the image pair;
performing a similarity-estimated affine transformation on the image pair to obtain a rigid registration result.

12. The method according to claim 11, wherein said performing an elastic registration on the image pair comprises:
performing a histogram equalization on the rigid registration result;
inputting the rigid registration result into a deep learning model to output an intermediate result by the deep learning model, wherein the intermediate result is a deformation field, wherein the deep learning model is trained using the image pair of the expression image-the image data;
performing nearest-neighbor interpolation transformation on the rigid registration result based on the deformation field to obtain an elastic registration result.

13. The method according to claim 5, wherein said performing registration on at least one of the expression data and the image data to generate at least one of an external contour and region contour of the biological tissue sample comprises:
registering expression data from two adjacent or proximate sections to obtain registered expression data;
generating at least one of the external contour and the region contour of the biological tissue sample based on the registered expression data.

14. The method according to claim 1, wherein said generating a three-dimensional display data of the biological tissue sample based on at least one of the external contour and the region contour comprises:
for the registration result obtained by performing registration on at least one of the expression data and the image data, performing bioinformatics analysis to obtain clustering and annotation results;
constructing a three-dimensional array based on the clustering and annotation results, and incorporating spatial scale information;
rendering with a three-dimensional rendering tool, to obtain the three-dimensional display data of the biological tissue sample.

15. (canceled)

16. (canceled)

17. (canceled)

18. (canceled)

19. (canceled)

20. An electronic device, comprising:
at least one processor; and
a memory communicatively connected to the at least one processor,
wherein the memory stores instructions executable by the at least one processor, and when the instructions executed by the at least one processor, the at least one processor execute the method according to claim 1.

21. A non-transitory computer-readable storage medium comprising computer executable instructions, wherein when the instructions are used for causing a computer to execute the method according to claim 1.

22. (canceled)

23. (canceled)

24. The method according to claim 5, further comprising:
obtaining a grayscale image and obtaining a histogram of the image data based on the grayscale image;
binarizing the image data using a maximum entropy threshold;
merging the binary image with the image data.
